(19) United States
(12) Patent Application Publication
(10) Pub. No.: US 2008/0076147 A1
(43) Pub. Date: Mar. 27, 2008

(54) BIOFILM SAMPLING DEVICE

(75) Inventors: Mohamed A. El-Azizi, New Cairo City (EG); Nancy Khardori, Springfield, IL (US)

Correspondence Address: SENNIGER POWERS LLP, ONE METROPOLITAN SQUARE, 16TH FLOOR, ST LOUIS, MO, 63102, US

(73) Assignee: BOARD OF TRUSTEES OF SOUTHERN ILLINOIS UNIVERSITY, Springfield, IL (US)

(21) Appl. No.: 11/861,737

(22) Filed: Sep. 26, 2007

Related U.S. Application Data
(60) Provisional application No. 60/847,497, filed on Sep. 26, 2006.

(51) Int. Cl. C12Q 1/24; C12M 1/14

(52) U.S. Cl. 435/30; 435/309.1

(57) ABSTRACT

An apparatus for forming biofilm on a fluid flow conduit, such as a medical device, enables in vitro simulation of biofilm formation to determine suitable treatments for minimizing or preventing biofilm formation in vivo.

### FIELD OF THE INVENTION

The present invention generally relates to an apparatus for forming biofilms on a fluid flow conduit, such as a medical device, to simulate in vivo conditions, and a method of testing the biofilm formed on such a conduit for colonized cells.

### BACKGROUND OF THE INVENTION

In recent years, extensive study of microbial growth, especially bacteria, has shown that they can form complex layers that adhere to surfaces, leading to problems in health care and food processing. This microbial property is important for implantable or insertable medical devices such as catheters and stents made of metallic, polymeric or a composite of metallic and polymeric materials, which frequently occlude due to microbial colonization and adhesion. This problem is particularly prevalent with medical devices that are adapted to remain implanted for a relatively long-term, i.e., from about 30 days to about 12 months or longer. Microbes such as bacteria often colonize on and around the medical device and, upon attaching to surfaces of the device, proliferate and form aggregates within a complex matrix consisting of extracellular polymeric substances, typically polysaccharides. The mass of attached microorganisms and the associated extracellular polymeric (glycocalyx) substances is commonly referred to as a biofilm. Antimicrobial agents have difficulty penetrating biofilms and killing and/or inhibiting the proliferation of the microorganisms within the biofilm. The colonization of the microbes on and around the device and the synthesis of the biofilm barrier eventually result in encrustation, occlusion and failure of the device. More importantly, biofilm formation on medical devices can lead to infection, sepsis or even death of a patient.

Biofilms on indwelling medical devices may be composed of gram-positive or gram-negative bacteria or yeasts. Bacteria commonly isolated from these devices include the gram-positive Enterococcus faecalis, Staphylococcus aureus, Staphylococcus epidermidis, and Streptococcus viridans; and the gram-negative Escherichia coli, Klebsiella pneumoniae, Proteus mirabilis, and Pseudomonas aeruginosa. Commonly encountered yeast species that can form biofilms include Candida albicans, Saccharomyces cerevisiae and Candida parapsilosis, Candida krusei, and Torulopsis glabrata. These organisms may originate from the skin of patients or health-care workers, tap water to which entry ports are exposed, or other sources in the environment. Biofilms may be composed of a single species or multiple species, depending on the device and its duration of use in the patient.

The importance of biofilm formation on medical devices is exemplified by coagulase negative staphylococci, S. epidermidis. This strain of bacteria was previously considered a non-pathogenic organism; however, it has emerged as the most common cause of foreign body infection and nosocomiai sepsis. It is the major cause of prosthetic valve endocarditis, vascular graft infection, artificial hip and knee infection, and catheter related sepsis. Although less virulent than S. aureus and many other bacteria, it is highly resistant to most antimicrobials except vancomycin and rifampin.

The process of biofilm formation is complex and influenced by many factors. Firstly, the microorganisms must adhere to the exposed surfaces of the device long enough to become irreversibly attached. The rate of cell attachment depends on the number and types of cells in the liquid to which the device is exposed, the flow rate of liquid through the device, and the physicochemical characteristics of the surface. Components in the liquid may alter the surface properties and also affect rate of attachment. Once these cells irreversibly attach and produce extracellular polysaccharides to develop a biofilm, rate of growth is influenced by flow rate, nutrient composition of the medium, antimicrobial-drug concentration, and ambient temperature.

Model systems were developed to study the biofilms on various indwelling medical devices. One type of device for monitoring biofilm buildup is described in the Canadian Journal of Microbiology (1981), Volume 27, pages 910-927, in which McCoy et al. describes the use of a so-called Robbins device. The Robbins device includes a tube through which water in a recycling circuit can flow. The tube has a plurality of ports within the tube wall, each port being provided with a removable stud, the stud having a biofoulable surface and being capable of being retained within the port in a fixed relationship with respect to the tube so that the biofoulable surface forms part of the internal surface of the tube. Each of the studs may be removed from the ports after a desired time interval and the surfaces analyzed for the growth of microorganisms. Alternatively, any surface growth may be removed and studied independent of the stud. The number of microorganisms can be estimated for instance by physical or chemical means, e.g. by detection of bacterial ATP or by further culturing of the microorganisms and analyzing the products.

A modified Robbins device (MRD) is constructed of an acrylic block 42 cm long with a lumen of 2 mm×10 mm. The MRD has 25 evenly spaced specimen plugs so that the catheter material (0.5 cm²) that is attached to the plugs lies flush with the inner surface without disturbing flow characteristics. The modified Robbins device is described in greater detail in Nickel, et. al. “Tobramycin resistance of Pseudomonas aeruginosa cells growing as a biofilm on urinary catheter material,” Antimicrobial Agents and Chemotherapy, 27:619-624 (1985). There is also a modified MRD, wherein the device was adapted to fit 2- to 3-mm segments of silicone or Teflon vascular catheters (see, e.g., Khardori et al., Journal of Infectious Diseases, Vol. 164, pp. 108-113, 1991).

However, additional systems for monitoring biofilm formation are needed, which are simple to operate, reusable and able to be sterilized. In addition, these systems should closely simulate the in vivo or in situ conditions for each device, while at the same time providing reproducible, accurate results.

### SUMMARY OF THE INVENTION

The present invention is directed to an apparatus for forming biofilms on a fluid flow conduit, such as a medical device.

In one apparatus embodiment, the apparatus comprises a tubular body defining a test chamber having a vertical axis, the body having upper and lower ends; an upper end closure closing the upper end of the tubular body; a lower end closure closing the lower end of the tubular body; an outlet in the tubular body toward the upper end of the body; and an opening in one of the end closures. The opening is for receiving a fluid flow conduit such that the conduit extends generally vertically into the test chamber to define an annular space between the conduit and the tubular body. Fluid passing through the conduit and into the chamber flows into the annular space and exits the outlet.

The present invention is also directed to a system which includes the apparatus of the invention, the fluid flow conduit, and associated components.

In a first system embodiment, the system comprises the apparatus, a fluid flow conduit in the opening in one of the end closures and extending into the test chamber, and at least one fluid source for delivery of a fluid to the conduit.

In a second system embodiment, the system comprises the apparatus, a fluid flow conduit in the opening in one of the end closures and extending into the test chamber, and at least two fluid sources, connected in series, for delivery of a fluid mixture to the conduit.

In a third system embodiment, the system comprises the apparatus wherein the opening is a first opening in the upper end closure, a fluid flow conduit in the first opening, and a second opening in the lower end closure through which fluid flowing continuously into the test chamber exits the test chamber.

In a fourth system embodiment, the system comprises the apparatus, and a fluid flow conduit in the first opening. The outlet is closed thereby to allow static testing of fluid in the chamber.

The present invention is also directed to a process for growing and assaying biofilms formed on a fluid flow conduit, such as a medical device.

In one process embodiment, the process comprises providing a system comprising an apparatus as set forth in claim 1, a fluid flow conduit in the opening in one of the end closures of the apparatus and extending into the test chamber, and at least one fluid; passing the fluid through the conduit and into the chamber such that it flows into the annular space, so as to form a biofilm on at least one surface of the conduit; removing the conduit from the apparatus; and analyzing the biofilm.

In any of the system or process embodiments as described above, the fluid source can include serum, saliva, blood, urine, a gas, an antibiotic, or a biofilm formation inhibitor. In some embodiments, the fluid source includes a glycoprotein, a polysaccharide, a non-steroidal anti-inflammatory drug (NSAID), tetracycline, rifamycin, a macrolide, penicillin, cephalosporin, a beta-lactam antibiotic, an aminoglycoside, chloramphenicol, a sulfonamide, a glycopeptide, a quinolone, fusidic acid, trimethoprim, metronidazole, clindamycin, mupirocin, a polyene, an azole, a benzalkonium halide, a silver salt, a beta-lactam inhibitor, triclosan, chlorhexidine, nitrofurazone, rifampin, gentamycin, minocyclin, imipenem, aztreonam, sulbactam, or a chelating agent. In some embodiments, the biofilm formation inhibitor comprises EDTA (ethylenediaminetetraacetic acid), EGTA (O,O′-bis(2-aminoethyl)ethyleneglycol-N,N,N′,N′-tetraacetic acid), salicylic acid or a salt thereof, mucin, or chitosan. In a preferred embodiment, the biofilm formation inhibitor is mucin. The fluid flow conduit can be a medical device, such as a catheter, a cannula, a vascular graft, a vascular catheter port, a vascular access device, a tube, a shunt, a heart valve, an incontinence device, or a penile implant. In some embodiments, the fluid flow conduit is a vascular catheter, a urinary catheter or an endotracheal tube. In some embodiments, the fluid flow conduit is treated with an antibiotic or a biofilm formation inhibitor, such as by coating it with a solution, before passing the fluid through the conduit to expose the conduit to biofilm forming organisms. The biofilm can be analyzed by any means available in the art. In some embodiments, the biofilm is physically removed from the conduit or at least one portion of the conduit and the removed biofilm is analyzed as desired. The biofilm, for example, can be analyzed for the number of colonized cells per unit of surface area of the conduit.

In any of the apparatus, system or process embodiments as described above, the apparatus can further include a seal on the one end closure for sealing around the conduit. The apparatus can also include a seal on the other end closure. The apparatus can comprise an opening in the other of the end closures whereby said fluid flow conduit can be selectively placed in either of the two openings, and a removable plug for closing the opening not selected. The opening can lie generally on the vertical axis of the chamber. A pump can be included for pumping fluid from a fluid source to the conduit. An outlet line can be included in the apparatus for delivering fluid from the outlet to a collection vessel. The apparatus can include a filter in the outlet line for filtering bacteria from the fluid. The fluid flow conduit can extend through either the upper end closure down into the test chamber, or through the lower end closure up into the test chamber.

Other objects and features will be in part apparent and in part pointed out hereinafter.

### BRIEF DESCRIPTION OF THE DRAWINGS

FIG. 1 is a schematic diagram of a system for formation of a biofilm on a fluid flow conduit fastened within an upper end of an apparatus of the invention.

FIGS. 2 and 2A are photographs of the apparatus and a catheter as the fluid flow conduit.

FIG. 3 is a schematic diagram of a system as in FIG. 1 further including a bacteria filter and a pump in the outlet line to remove bacteria from the effluent.

FIG. 4 is a schematic diagram of a system for formation of a biofilm on a fluid flow conduit fastened within a lower end of an apparatus of the invention.

FIG. 5 is a schematic diagram of a system as in FIG. 4 further including a bacteria filter and a pump in the outlet line to remove bacteria from the effluent.

FIGS. 6 and 7 are each schematic diagrams of a system for formation of a biofilm on a fluid flow conduit fastened within an upper end of an apparatus of the invention wherein two fluid sources are connected in series for delivery of a fluid mixture to the conduit.

FIG. 8 is a schematic diagram of a system for formation of a biofilm on a fluid flow conduit fastened within an upper end of an apparatus of the invention wherein the outlet is closed thereby to allow static testing of fluid in the chamber.

FIG. 9 is a schematic diagram of a system for formation of a biofilm on a fluid flow conduit fastened within an upper end of an apparatus of the invention wherein fluid flows continuously into the test chamber and exits the test chamber through a second opening in the lower end closure.

FIGS. 10A-B are graphs depicting the biocide concentration (in ppm) required to kill 100% of the Acinetobacter baumanii bacteria present as planktonic cells or 50% of bacteria that grew in the biofilm within a period of time ranging from 5 to 60 minutes.

FIGS. 11A-B are graphs depicting the biocide concentration (in ppm) required to kill 100% of the Burkholderia cepacia bacteria present as planktonic cells or 50% of bacteria that grew in the biofilm within a period of time ranging from 5 to 60 minutes.

FIGS. 12A-B are graphs depicting the biocide concentration (in ppm) required to kill 100% of the Escherichia coli bacteria present as planktonic cells or 50% of bacteria that grew in the biofilm within a period of time ranging from 5 to 60 minutes.

FIGS. 13A-B are graphs depicting the biocide concentration (in ppm) required to kill 100% of the Enterococcus faecalis bacteria present as planktonic cells or 50% of bacteria that grew in the biofilm within a period of time ranging from 5 to 60 minutes.

FIGS. 14A-B are graphs depicting the biocide concentration (in ppm) required to kill 100% of the MRSA bacteria present as planktonic cells or 50% of bacteria that grew in the biofilm within a period of time ranging from 5 to 60 minutes.

FIGS. 15A-B are graphs depicting the biocide concentration (in ppm) required to kill 100% of the Staphylococcus epidermis bacteria present as planktonic cells or 50% of bacteria that grew in the biofilm within a period of time ranging from 5 to 60 minutes.

FIGS. 16A-B are graphs depicting the biocide concentration (in ppm) required to kill 100% of the Pseudomonas aeruginosa bacteria present as planktonic cells or 50% of bacteria that grew in the biofilm within a period of time ranging from 5 to 60 minutes.

FIGS. 17A-B are graphs depicting the biocide concentration (in ppm) required to kill 100% of the Stenotrophomonas maltophilia bacteria present as planktonic cells or 50% of bacteria that grew in the biofilm within a period of time ranging from 5 to 60 minutes.

FIG. 18 is a graph depicting the number of MRSA colony forming units per unit of surface area (CFU/cm²) on an endotracheal tube when treated with various concentrations of mucin.

FIG. 19 is a graph illustrating the number of MRSA colony forming units per unit of surface area (CFU/cm²) on the tube for various endotracheal tubes which are albumin coated or uncoated.

Corresponding reference characters indicate corresponding parts throughout the drawings.

### DESCRIPTION OF THE PREFERRED EMBODIMENTS

The present invention enables a clinical physician, a research scientist or other medical personnel to simulate a patient's condition in vitro with regard to formation of a biofilm on a medical device or any material that is implanted or inserted within the body of a patient. A catheter, endotracheal tube or other fluid flow conduit can be inserted into the apparatus of the invention and tested under various fluid flow conditions which simulate the in vivo environment in which biofilms form. Potential treatments for preventing or minimizing biofilm formation and its consequent risk of infection can be tested in vitro using the apparatus and method of the invention. More specifically, a fluid inoculated with a pathogen which mimics the actual in vivo environment in which the conduit is used flows through the conduit and into the test chamber of the apparatus to surround the conduit in the fluid. The fluid flow rate is selected so as to mimic conditions of actual use as well. Over a period of hours or days, a biofilm forms on the conduit which can be dislodged from the conduit and analyzed for its bacterial cell content. Antibiotics or biofilm formation inhibitors can be mixed with the fluid at various concentrations to determine effective treatments for a patient. Alternatively, the conduit can be pretreated (e.g., coated) with antibiotics or biofilm formation inhibitors and then subjected to fluid flow to determine the effectiveness of the pretreatment. The apparatus and method can be used, for example, in modeling biofilm formation on an endotracheal tube used in ventilating intensive care patients to determine effective treatments for the prevention of ventilator associated pneumonia.

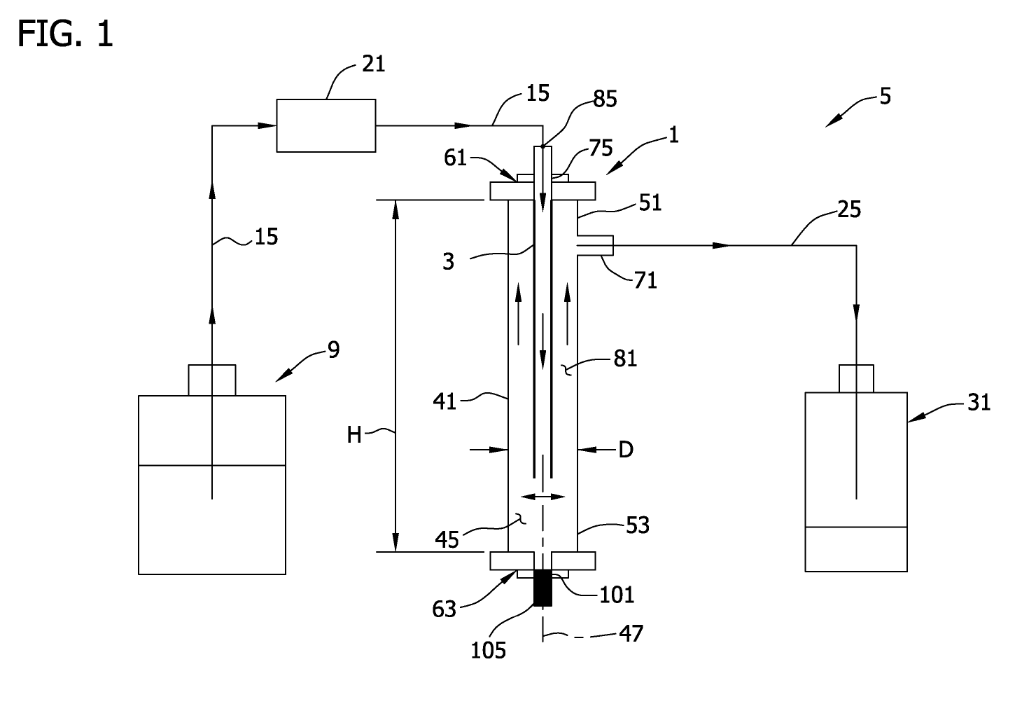

One aspect of the present invention is directed to an apparatus used in forming biofilms. Referring now to the drawings, and first more particularly to FIG. 1, sampling apparatus of this invention is designated in its entirety by the reference numeral 1. The apparatus 1 is especially suited for sampling biofilm formed on a fluid flow conduit 3. The conduit may be a catheter, an endotracheal tube, or other medical device. The apparatus 1 can be used in a variety of sampling systems and configurations, as will be described. FIG. 1 illustrates an exemplary open, continuous flow system, generally designated 5, comprising a fluid source 9, a feed line 15 connecting the fluid source to the fluid flow conduit 3, and a pump 21 for pumping fluid from the source to the conduit. The fluid source 9 may be a sealed or unsealed receptacle containing a fluid such as saliva, serum, blood, urine, any human or animal body fluid, or any artificial medium or solution which can mimic the body fluid (e.g., artificial saliva) or which is physiologically acceptable when administered to a human or animal, for example. Such fluids are commercially available. The system also includes an outlet line 25 for delivering fluid from the sampling apparatus 1 to a suitable receptacle such as a sealed or unsealed collection vessel 31.

As shown in FIG. 1, the sampling apparatus 1 comprises a tubular body 41 defining a test chamber 45 having a central vertical axis 47. The size of the test chamber 45 will vary. By way of example, the height H of the test chamber may be in the range of about four inches to about 20 inches; the diameter D of the test chamber (corresponding to the internal diameter of the tubular body) may be in the range of about 0.3 inches to about 1 inch; and the volume of the test chamber may be in the range of about 5 mL to about 20 mL. The body 41 is preferably circular in horizontal cross section, but it may have other shapes (e.g., polygonal) without departing from the scope of this invention. The body 41 has upper and lower ends designated 51 and 53, respectively. An upper end closure 61 is provided for closing the upper end 51 of the tubular body 41, and a lower end closure 63 is provided for closing the lower end 53 of the tubular body. These end closures 61, 63 preferably have releasable and sealing connections (e.g., threaded connections) with respective ends of the body 41, as by threaded connections. Alternatively, one of the end closures may be secured permanently in place. The tubular body 41 has an outlet 71 toward its upper end 51, i.e., in its upper half, and preferably adjacent its upper end. Alternatively, the outlet 71 may be in the upper end closure 61.

An opening 75 is provided in the upper end closure 61 for receiving the fluid flow conduit 3 such that the conduit 3 extends generally vertically down into the test chamber 45 and terminates short of the opposite (lower) end closure 63, an annular space 81 thus being defined between the exterior surface of the conduit 3 and the inside surface of the tubular body 41. The conduit 3 has a relatively snug, sliding fit in the opening 75. Optionally, a seal (not shown) may be provided on the upper end closure 61 for sealing around the conduit 3 to prevent leakage through the opening 75. As shown in FIG. 1, the opening 75 is generally co-axial with the vertical axis 47 of the test chamber 41, but it will be understood that the opening may be off-center. In any case, the arrangement is such that fluid pumped from the fluid source 9 through the conduit 3 into the chamber 45 flows into the annular space 81 and, after filling the chamber to the level of the outlet 71, exits the outlet for delivery to the collection vessel 31. The feed line 15 has a releasable connection 85 with the conduit 3.

Optionally, the temperature of the tubular body 41 is controlled by suitable means, such as a heating mechanism and/or a cooling mechanism.

An opening 101 is also provided in the lower end closure 63 for receiving a fluid flow conduit when the sampling apparatus 1 is set up to operate in a different configuration, as will be described later. This opening is preferably (but not necessarily) co-axial with the vertical axis 47 of the test chamber and aligned with the opening 75 in the upper end closure 61. A removable closure 105 (e.g., plug) is provided for closing and sealing this opening 101 when it is not is use. The closure 105 has a releasable (e.g., threaded) and sealing connection with the lower end closure 63. Optionally, a seal (not shown) may be provided on the lower end closure 63 for sealing around the conduit 3 in the opening (when the plug 105 is removed) to prevent leakage.

The various parts of apparatus 1 may be fabricated from suitable materials, such as stainless steel, pyrex glass, or other sterilizable materials which do not impact the formation of a biofilm, and are preferably easy to handle and lightweight.

The apparatus 1 is held in position by a suitable support (not shown). The support may be a free-standing support with a clamp for releasably holding the tubular body in position, or a support which is fastened to another surface, such as bench or a wall.

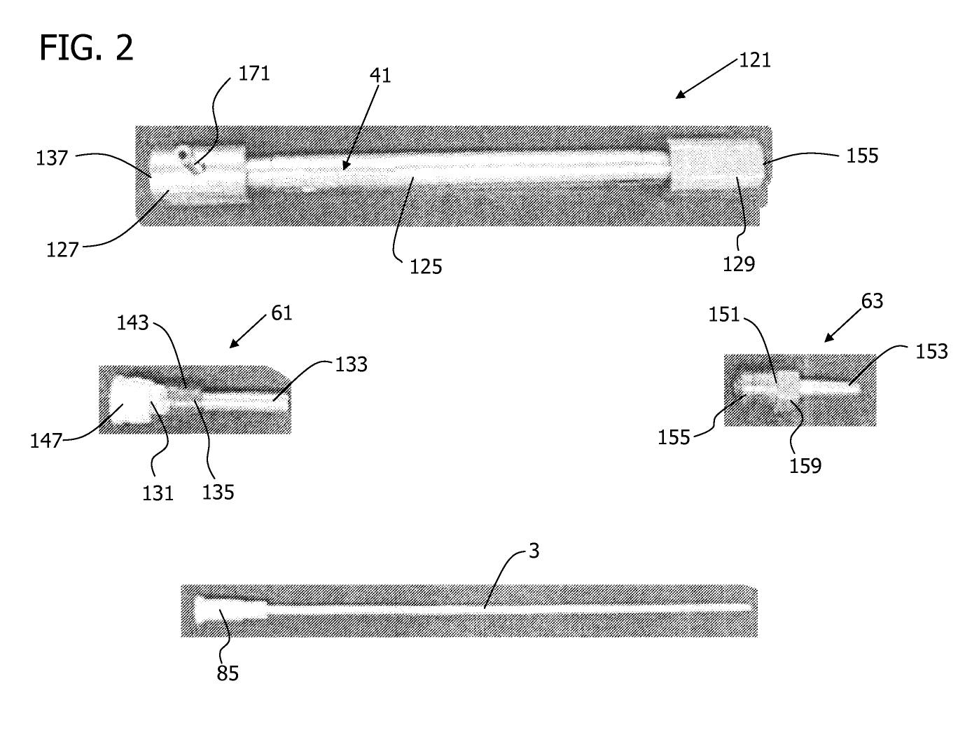

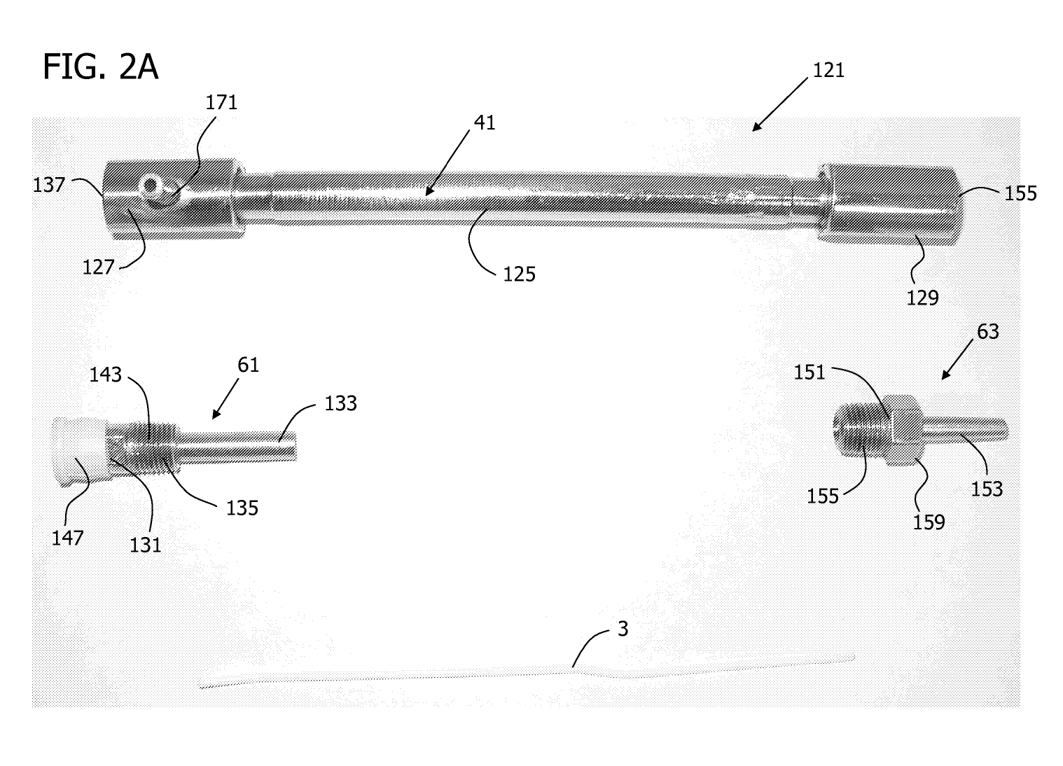

FIGS. 2 and 2A show a different embodiment of the sampling apparatus, generally indicated at 121. Apparatus 121 is similar to the apparatus 1 previously described and corresponding parts are indicated by corresponding reference numbers. In this embodiment, the tubular body 41 comprises multiple parts, including a central tube 125, an upper tubular fitting 127 affixed to the upper end of the central tube, and a lower tubular fitting 129 affixed to the lower end of the central tube. The upper end closure 61 comprises an upper portion 131 and a co-axial lower portion 133. A bore (not shown but corresponding to opening 75 of apparatus 1) extends through the upper and lower portions for receiving the conduit 3. The upper portion 131 is formed with external threads at 135 which mate with internal threads (not shown) in an opening 137 in the upper tubular fitting 127 of the tubular body 41. The exterior of the upper portion 131 is formed with flats 143 to facilitate turning of the end closure 61 to tighten and loosen it in the opening in the upper tubular fitting 127. When the upper end closure 61 is threaded in place, the lower sleeve-like portion 133 extends down a distance into the test chamber 45 generally along the axis of the test chamber for providing additional support to the conduit 3. A removable closure comprising a cap 147 fits over the upper portion 131 of the end closure 61 to close and seal the bore through the end closure, as when the apparatus 1 is not in use and/or when the apparatus is being used in certain flow configurations of the apparatus, as described below.

Still referring to FIGS. 2 and 2A, the lower end closure 63 of prototype 121 comprises an upper portion 151 and a lower portion 153. The upper portion is formed with external threads 155 which mate with internal threads (not shown) in an opening 155 in the lower tubular fitting 129. The exterior of the upper portion 151 is formed with flats 159 to facilitate turning of the end closure 63 to tighten and loosen it in the opening 155 in the lower tubular fitting 129. A bore (not shown but corresponding to opening 101 of apparatus 1) extends through the upper and lower portions 151, 153 of the lower end closure 63 generally co-axial with the test chamber 45. A removable closure comprising a cap (not shown) fits over the lower portion 153 of the end closure 63 to close and seal the bore through the lower end closure, as when the apparatus 1 is not in use and/or when the apparatus is being used in certain flow configurations of the apparatus, as described below.

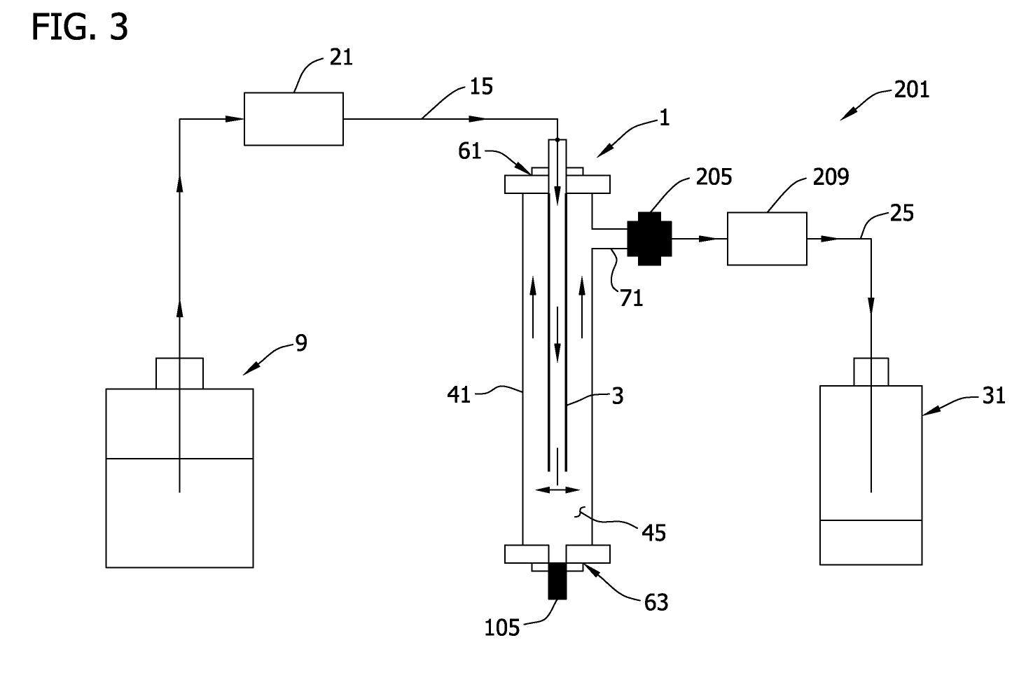

FIG. 3 illustrates use of the sampling apparatus 1 in a closed, continuous flow system, generally designated 201. This system is similar to the system shown in FIG. 1 and corresponding parts are indicated by the same reference numbers. In the system 201, a filter 205 is provided in the outlet 71 of the sampling apparatus for filtering bacteria from the fluid as it exits the test chamber. Alternatively, the filter 205 can be placed in the outlet line 25. A second pump 209 is provided downstream from the filter 205 for pumping filtered fluid to the collection vessel 31.

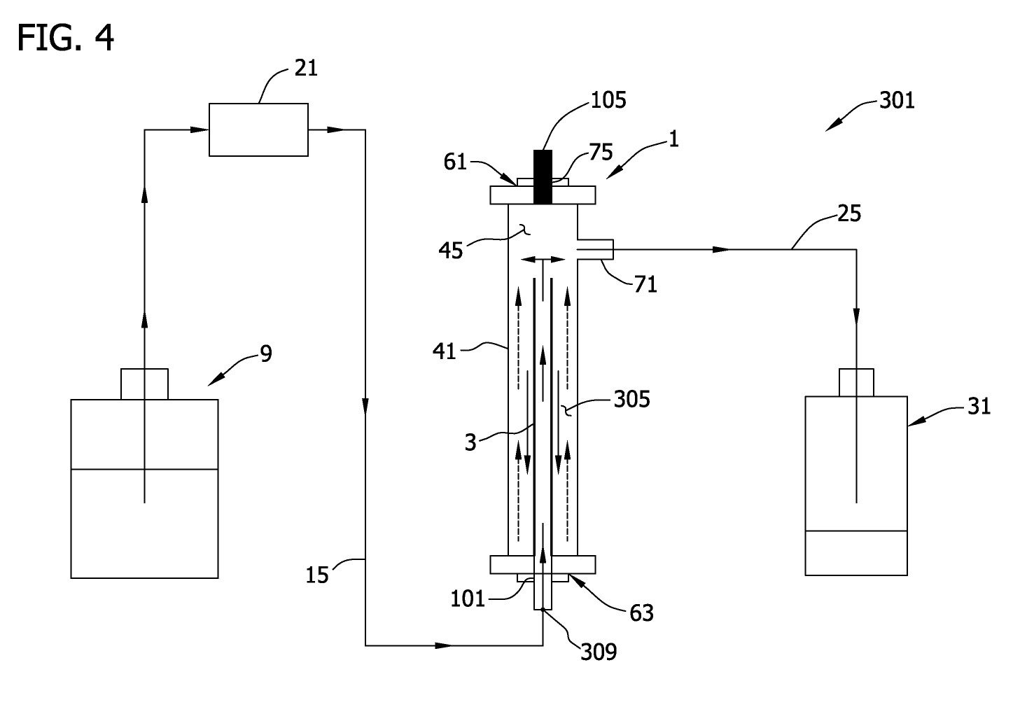

FIG. 4 illustrates use of the sampling apparatus 1 in an open, continuous flow system, generally designated 301, in which fluid flows into the test chamber 45 against the force of gravity. This system is similar to the system shown in FIG. 1 and corresponding parts are indicated by the same reference numbers. In the system 301, the plug 105 is removed from the opening 101 in the lower end closure 63 and the opening 75 in the upper end closure 61 is closed and sealed using plug 105 (or a different closure). The conduit 3 is placed in the lower opening 101 such that it extends vertically up into the test chamber 45 and terminates short of the upper end closure 61, an annular space 305 thus being defined between the exterior surface of the conduit 3 and the inside surface of the tubular body 41. The arrangement is such that fluid pumped from the fluid source 9 through the conduit 3 into the chamber 45 flows into the annular space 305 and, after the chamber has filled to the level of the outlet 71, exits the outlet for delivery to the collection vessel 31. The conduit has a releasable connection 309 with the feed line 15 similar to the connection 85 described above.

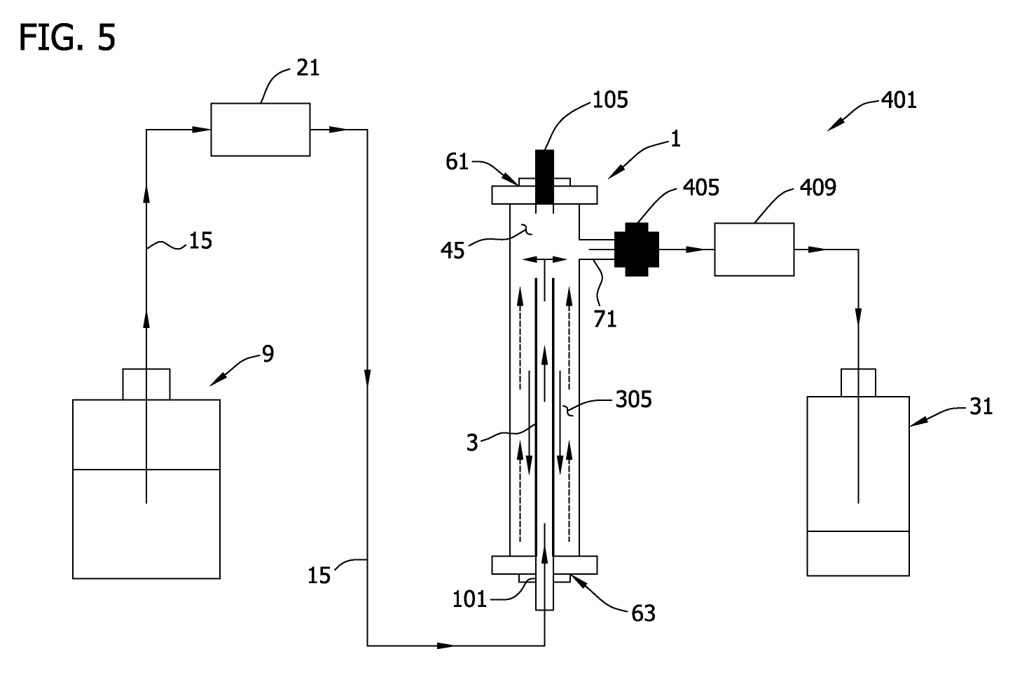

FIG. 5 illustrates use of the sampling apparatus 1 in a closed, continuous flow system, generally designated 401, in which fluid flows into the test chamber 45 against the force of gravity. This system is similar to the system shown in FIG. 4 and corresponding parts are indicated by the same reference numbers unless otherwise indicated. In the system 401, a filter 405 is provided in the outlet 71 of the sampling apparatus for filtering bacteria from the fluid as it exits the test chamber 45. Alternatively, the filter 405 can be placed in the outlet line 25. A second pump 409 is provided downstream from the filter 405 for pumping filtered fluid to the collection vessel 31.

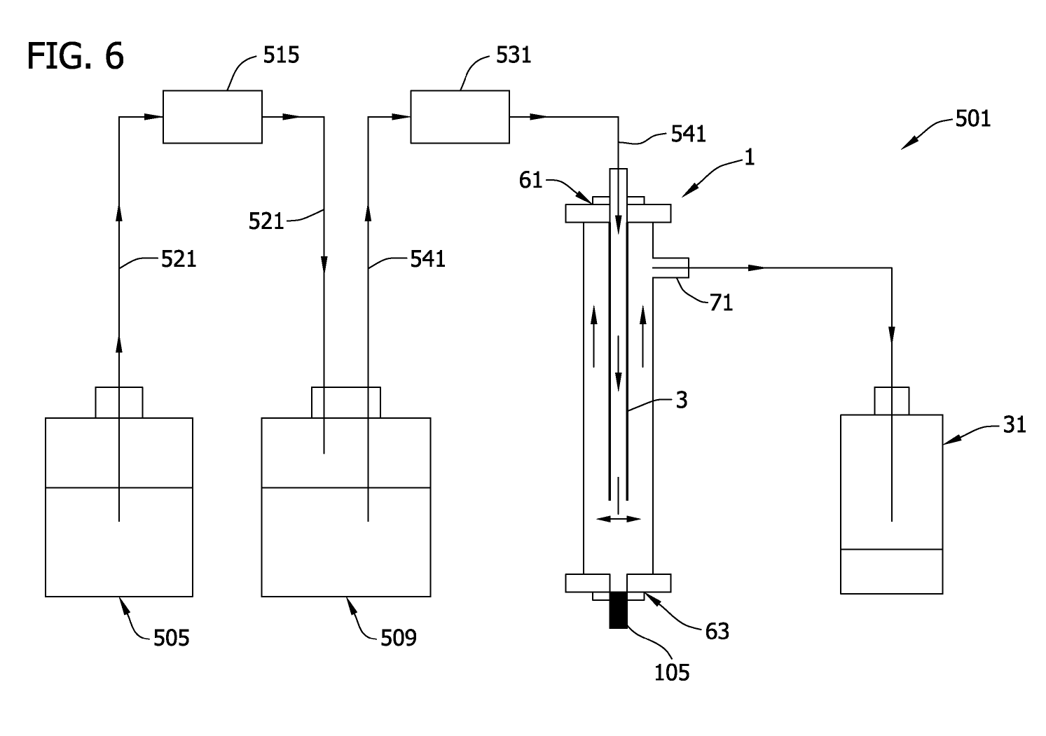

FIG. 6 illustrates use of the sampling apparatus 1 in a multi-source, open, continuous flow system, generally designated 501. This system is similar to the system shown in FIG. 1 and corresponding parts are indicated by the same reference numbers. In the multi-source system 501, the single fluid source 9 of FIG. 1 is replaced by two or more fluid sources, such as the first and second fluid sources indicated at 505 and 509 in FIG. 6. By way of example, the first fluid source 505 may comprise a sealed or unsealed vessel containing saliva, serum, blood, or urine, and the second fluid source 509 may comprise a sealed or unsealed vessel containing a suitable antibiotic. Fluid from the first fluid source 505 is pumped (using a first pump 515) through a first feed line 521 to the second source 509 where the two fluids mix. The fluid mixture is then delivered (using a second pump 531) through a second feed line 541 to the conduit 3 positioned in the sampling apparatus 1.

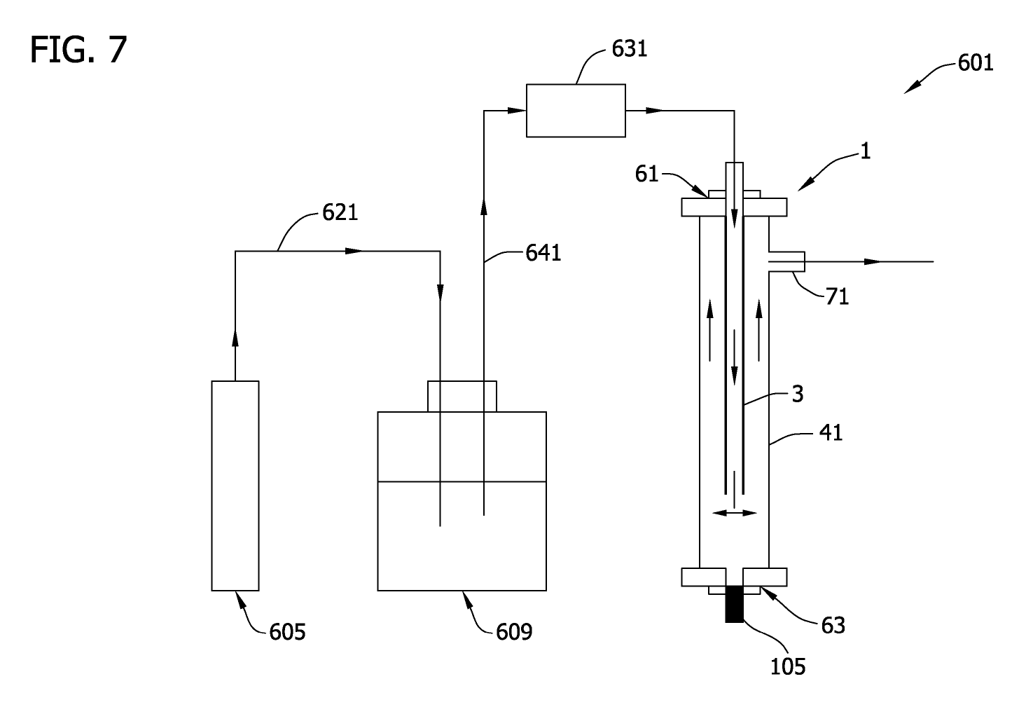

FIG. 7 illustrates use of the sampling apparatus 1 in another multi-source, open, continuous flow system, generally designated 601. This system is similar to the system shown in FIG. 1 and corresponding parts are indicated by the same reference numbers. In the multi-source system 601, the single fluid source 9 of FIG. 1 is replaced by two or more fluid sources, such as the first and second fluid sources indicated at 605 and 609 in FIG. 7. By way of example, the first fluid source 605 may comprise a sealed vessel containing oxygen or other gas (typically under pressure), and the second fluid source 609 may comprise a sealed vessel containing saliva or other fluid. Oxygen is delivered from the first fluid source 605 through a first feed line 621 to the second source 609 where the two fluids mix. The fluid mixture is then delivered (using pump 631) through a second feed line 641 to the conduit 3 positioned in the sampling apparatus 1.

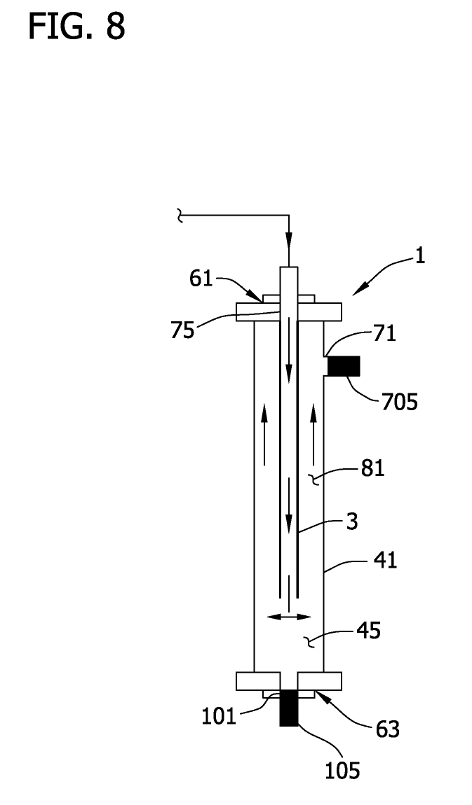

FIG. 8 illustrates use of the sampling apparatus 1 in a static flow configuration. The apparatus is essentially the same as the apparatus described above in regard to the first embodiment (FIG. 1), and corresponding parts are indicated by corresponding reference numbers. In the configuration of FIG. 8, the conduit 3 extends down into the test chamber 41 through the opening 75 in the upper end closure 61; the opening 101 in the lower end closure 63 is closed by the removable closure 105; and the outlet 71 of the tubular body 41 is closed by a second removable closure (e.g., plug 705). The arrangement is such that a predetermined quantity (volume) of fluid is delivered to the conduit 3 for flow into the test chamber 45 to fill the chamber to a predetermined level (e.g., substantially completely filled). The fluid is maintained in a static condition in the chamber 45 in contact with interior and exterior surfaces of the conduit for a predetermined period of time, after the end of which the conduit is removed and the fluid emptied from the test chamber by removing either or both of the closures 105, 705.

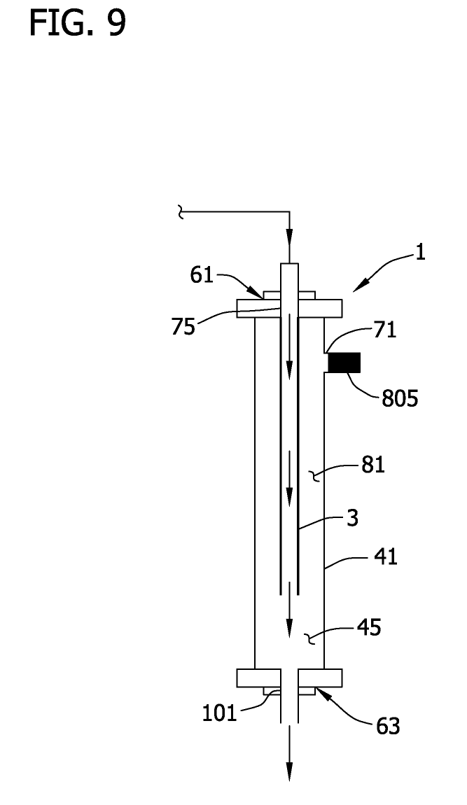

FIG. 9 illustrates use of the sampling apparatus 1 in an alternative continuous flow configuration. The apparatus 1 is essentially the same as the apparatus described above in regard to the first embodiment (FIG. 1), and corresponding parts are indicated by corresponding reference numbers. In the configuration of FIG. 9, the conduit 3 extends down into the test chamber 45 through the opening 75 in the upper end closure 61; the opening 101 in the lower end closure 63 is open (i.e., not plugged); and the outlet 71 of the tubular body 41 is closed by a removable closure 805. The arrangement is such that fluid is continuously delivered to the conduit 3 for flow into the test chamber 45 and then out of the test chamber 45 via opening 101, without filling the test chamber or substantial contact of the fluid with the exterior surface of the conduit 3.

The apparatus and system as described above is used for formation of biofilm on a fluid flow conduit. An embodiment of the present invention is directed to a process for growing and assaying biofilms on a test device in vitro. A system is provided which comprises an apparatus of the invention, a fluid flow conduit in the opening in one of the end closures of the apparatus and extending into the test chamber, and at least one fluid. The fluid passes through the conduit into the chamber such that it flows into the annular space, so as to form a biofilm on at least one surface of the conduit. The conduit is removed from the apparatus and analyzed for the formation of biofilm thereon.

The fluid selected for use in the process of the invention is that which most closely simulates the environment in which the conduit is used in the body. In preferred embodiments, the fluid comprises saliva, serum, blood, or urine. For example, the fluid can be saliva saturated in oxygen if the conduit is an endotracheal tube. If the conduit is a vascular catheter, the fluid can be serum or blood, while urine is a suitable fluid for evaluation of a urinary catheter or other urinary device.

When the conduit is used in vivo in a static environment, the conduit is in the first opening, and the outlet is closed to allow static testing of fluid in the chamber.

In some cases, only the interior of the conduit is exposed to the fluid during in vivo use, such as with some prosthetic devices which are in direct contact with blood. This condition can be modeled when the opening is a first opening in the upper end closure, the conduit is in the first opening, and the fluid flows into the test chamber and exits the test chamber through a second opening in the lower end closure.

The biofilm can be removed from the conduit or a portion of the conduit by any means known in the art, such as physical or chemical means. In one embodiment of the invention, the process further includes cutting the conduit into segments after the conduit is removed from the apparatus, placing the segments into a container; adding a buffer solution, such as saline solution, to the container, and physically removing the biofilm via sonication and vortexing. The removed biofilm is then assayed to determine the number of organisms present per unit of conduit surface area.

The fluid flow conduit of the invention can be any medical device or material exposed to fluid flow within the body of a human or animal and subject to formation of bacterial contamination. In one embodiment, the medical device that is tested for biofilm formation using the apparatus described herein is a catheter. In a preferred embodiment, the catheter is a vascular catheter such as a central venous catheter, or a urinary catheter. In another embodiment, the medical device is an endotracheal tube.

It is known that bacteria in the form of biofilms are more resistant to antimicrobial agents than are planktonic bacteria. Thus, medical devices may be coated or impregnated with a variety of different agents in order to determine their susceptibility to biofilm formation following such modification. For example, approaches that are contemplated herein include the use of low surface energy materials such as Teflon® and the use of surface coatings. Additionally, the medical devices such as catheters may be coated or impregnated with at least one antimicrobial agent and/or at least one biofilm formation inhibitor.

Antimicrobial agents include, but are not limited to, triclosan, chlorhexidine, nitrofurazone, benzalkonium chlorides, silver salts, antibiotics and combinations thereof. Classes of antibiotics used include tetracyclines, rifamycins, macrolides, penicillins, cephalosporins, other beta-lactam antibiotics (i.e. imipenem, aztreonam), aminoglycosides, chloramphenicol, sulfonamides, glycopeptides, quinolones, fusidic acid, trimethoprim, metronidazole, clindamycin, mupirocin, polyenes, azoles and beta-lactam inhibitors (i.e. sulbactam). The rifamycins are a group of antibitoics which are synthesized either naturally by the bacterium Amycolatopsis mediterranei, or artificially. There are at least seven rifamycins, namely Rifamycin A, B, C, D, E, S and SV. The rifamycin class also includes derivatives thereof such as rifampicin, rifabutin and rifapetine. Macrolides include erythromycin, azythromycin, clarithromycin, dirithromycin and roxithromycin. Penicillin group of antibiotics includes benzathine benzylpenicilline, benzylpenicillin (penicilline G), phenoxymethylpenicillin (penicillin V), and semi-synthetic penicillins including methicillin, dicloxacillin, flucloxacillin, oxacillin, amoxicillin, ampicillin, piperacillin, ticarcillin, azlocillin and carbenicillin. Cephalosporin antibiotics include cefcapene, cefdaloxime, cefdinir, cefetamet, cefixime, cefmenoxime, cefodizime, cefoperazone, cefotaxime, cefpimizole, cefpodoxime, cefteram, ceftibuten, ceftiofur, ceftiolene, ceftizoxime, ceftriaxone, ceftazidime, cefpiramide, cefsulodin, cefclidine, cefepime, cefluprenam, cefoselis, cefozopran, cefpirome and cefquinome. Aminoglycosides include but are not limited to amikacin, gentamycin, kanamycin, tobramycin, neomycin, netilmicin and streptomycin. Sulfonamide antibiotics (sulfa drugs) include prontosil, sulfadiazine, sulfamethizole, sulfamethoxazole, sulfasalazine, sulfisoxazole, and various high-strength combinations of the latter three sulfonamides. Quinolones include but are not limited to cinoxacin, flumequine, nalidixic acid, oxolinic acid, piromidic acid, pipemidin acid, ciprofloxacin, enxacin, fleroxacin, levofloxacin, lomefloxacin, nadifloxacin, norfloxacin, ofloxacin, pefloxacin, rufloxacin, balofloxacin, gatifloxacin, grepafloxacin, pazufloxacin, sparfloxacin, temafloxacin, tosufloxacin, clinafloxacin, gemiflxacin, moxifloxacin and sitafloxacin.

Preferably, antibiotics are selected from the groups of rifamycin, microlides, quinolones and penicillins. More preferably, the antibiotics that may be used to coat or impregnate a medical device include but are not limited to clindamycin, gentamycin, neomycin, kanamycin, ciprofloxacin, mupirocin, vancomycin and penicillin.

Microbial attachment/biofilm synthesis inhibitors include, but are not limited to, glycoproteins such as mucin, polysaccharides such as chitosan, non-steroidal anti-inflammatory drugs (NSAIDs) and chelating agents such as EDTA (ethylenediaminetetraacetic acid), EGTA (O,O′-bis(2-aminoethyl)ethyleneglycol-N,N,N′,N′-tetraacetic acid) and mixtures thereof. Among preferred NSAIDs are salicylic acid and salts or derivatives thereof. Preferred salts of salicylic acid include, but are not limited to, sodium salicylate and potassium salicylate.

Thus, one skilled in the art can readily apply any of the possible single coating and/or impregnating agents or combinations of such agents to a medical device in order to determine its susceptibility towards biofilm formation. By way of example, a medical device may be coated with an antimicrobial agent such as triclosan or with a biofilm synthesis inhibitor such as salicylic acid. In another embodiment, a combination of two or more antimicrobial agents such as triclosan and penicillin or triclosan and clindamycin may be used. Combinations of two or more antibiotics can also be applied. Such combinations include, e.g., ampicillin and ciproflxacin, gentamicin and ciprofloxacin, imipenem and tobramycin, cefodizime and vancomycin, penicillin and streptomycin, and rifamycin and erythromycin.

In another embodiment, a combination of at least one antimicrobial agent such as tetracycline and a microbial attachment inhibitor such as salicylic acid can be coated or impregnated onto a medical device. Alternatively, a combination of two or more microbial attachment inhibitors, such as a salicylic acid and EDTA can be applied to a medical device. One skilled in the art can readily determine and apply any of a number of different combinations of possible antimicrobial agents and/or biofilm formation inhibitors that can be used on medical devices. Furthermore, methods for coating or impregnating the medical devices with agents mentioned above are well known in the art. For example, one method to coat a medical device is to simply flush the surface of the device with a solution of antibiotics.

In addition to coating or impregnating antibiotics into medical devices, an antimicrobial agent and/or biofilm formation inhibitor can be mixed with the fluid that is run through the medical device being tested in the apparatus regardless of whether the medical device is coated or uncoated. In one embodiment, a single antibiotic or biofilm formation inhibitor is included in the fluid. Preferably, the antibiotic is selected from ciprofloxacin, vancomycin, neomycin, tobramycin, ampicillin, amoxicillin, rifamycin, oxacillin, cefodizime and gentamycin. In another embodiment, a combination of two or more antibiotics and/or biofilm formatin inhibitors is included in the fluid. By way of example, combinations of antibiotics include but are not limited to vancomycin and ampicillin, ciprofloxacin and gentamycin, cefodizime and oxacilline, rifamycin and imipenem, and amoxicillin and mupirocin.

The amount of the antimicrobial agent and/or biofilm formation inhibitor used depends on a number of factors including the selected antibiotic(s), length of time for testing the medical device, use and duration of use of said device, rate at which the antibiotic flows through the device, and microbes involved in biofilm formation.

The present invention is useful in determining the biofilm formation of any microbes that are capable of attaching to a medical device. In one embodiment, the microbes are selected from bacteria, yeast and fungi. In a preferred embodiment, the microbes capable of biofilm formation are bacteria.

In another preferred embodiment, bacteria are selected from the gram-positive Enterococcus faecalis, Staphylococcus aureus, Staphylococcus epidermidis, and Streptococcus viridans; and the gram-negative Escherichia coli, Klebsiella pneumoniae, Proteus mirabilis, and Pseudomonas aeruginosa. In still another preferred embodiment, the bacteria are Staphylococcus epidermidis. In another embodiment, yeast are selected from Candida albicans and Saccharomyces cerevisiae.

### Definitions

The term “antimicrobial agent” as used herein means a substance that kills and/or inhibits the proliferation and/or growth of microbes, particularly bacteria, fungi and yeast. Antimicrobial agents, therefore, include biocidal agents and biostatic agents as well as agents that possess both biocidal and biostatic properties.

By “biofilm” is meant the mass of microorganisms attached to a surface, such as a surface of a medical device, and the associated extracellular substances produced by one or more of the attached microorganisms. The extracellular substances are typically polymeric substances and commonly comprise a matrix of complex polysaccharides, proteinaceous substances and glycopeptides. This matrix or biofilm is also commonly referred to as slime or glycocalyx.

“Medical devices” are herein defined as any device, biomaterial or ancillary which may be inserted or implanted into a human being or other animal, or placed at the insertion or implantation site (such as the skin near the insertion or implantation site), and which include at least one surface which is susceptible to colonization by biofilm embedded microorganisms. Such devices include, for example, disposable, reusable or permanent devices such as catheters, (e.g., central venous catheters, dialysis catheters, long-term tunneled central venous catheters, short-term central venous catheters, peripherally inserted central catheters, peripheral venous catheters, pulmonary artery Swan-Ganz catheters, urinary catheters, and peritoneal catheters), long-term urinary devices, tissue bonding urinary devices, vascular grafts, vascular catheter ports, wound drain tubes, ventricular catheters, hydrocephalus shunts, heart valves, heart assist devices (e.g., left ventricular assist devices), pacemaker capsules, incontinence devices, penile implants, small or temporary joint replacements, urinary dilators, cannulas, elastomers, hydrogels, surgical instruments, dental instruments, tubings (e.g., intravenous tubes, breathing tubes, endotracheal tubes, typanostomy tubes, dental water lines, dental drain tubes, and feeding tubes), fabrics, paper, indicator strips (e.g., paper indicator strips or plastic indicator strips), adhesives (e.g., hydrogel adhesives, hot-melt adhesives, or solvent-based adhesives), bandages, and orthopedic implants.

The term “microbial attachment inhibitor” is used interchangeably with the term “biofilm formation inhibitor” or “biofilm synthesis inhibitor” and refers to an agent that inhibits the attachment of microbes onto and the synthesis and/or accumulation of biofilm on a surface of an implantable or insertable medical device.

Having described the invention in detail, it will be apparent that modifications and variations are possible without departing from the scope of the invention defined in the appended claims.

### EXAMPLES

The following non-limiting examples are provided to further illustrate the present invention.

### Example 1

### Evaluation of Bacterial Resistance in Planktonic Cell and Biofilm Phases in Vascular Catheter Models

A system as shown in FIG. 6 was used in this study. Serum and a biocide were placed in sealed bottles as the first fluid source 505 and the second fluid source 509, respectively. The fluid sources were connected to a pump 515 by conventional IV tubing. A second pump 531 was used to deliver the fluid mixture to the vascular catheter 3 positioned in the sampling apparatus 1.

Initially, only the first fluid source, serum inoculated with Acinetobacter baumanii (10×10⁹ CFU/ml), was used to contaminate the catheter with planktonic cells. The inoculated fluid continuously flowed through IV tubing directly through the catheter for 120 minutes to form a layer of planktonic cells on the catheter. Fluid flow was then stopped and the tubing was then reconfigured as shown in FIG. 6 so that the second fluid source could be introduced. In these test runs, the first fluid source was inoculated with the same concentration of bacteria, and the second fluid source was a biocide (16-16,000 ppm) selected from hydrogen peroxide (H₂O₂), sodium hypochlorite (SHC), peracetic acid (PAA) and gluteraldehyde (GLT). The fluid mixture continuously flowed through the catheter for 5, 10, 15, 30, 45 or 60 minutes before fluid flow was discontinued to determine bacterial resistance of the planktonic cells on the catheter. In all, six runs were completed for each of four different biocides. After each run, the test catheter was then cut aseptically into 1 cm segments. The segments were transferred to test tubes containing one ml saline and the adherent cells were dislodged by sonication in an aquasonic device, vortexed for about one minute, and then counted on Tropic soya Agar. The number of viable cells were determined for each catheter and were compared to the number of viable cells in control samples (i.e., biocide-free serum) to calculate the biocide concentration required to kill 50% of the bacteria in the biofilm at the same time interval. Instead of saline, other appropriate buffer solutions can be used. In addition to plating cells on agar and counting the number of colonies, the amount of biofilm formed can be determined by a semiquantitive microtiter plate method. For example, adherent cells can be dislodged from test catheters as described above. The aliquots of solutions containing the cells from each catheter piece can then be added separately to wells of a 96-well tissue culture plate and incubated at 37° C. for 24 h. After several (e.g., three) washes with PBS, any remaining biofilm is stained with safranin O dye for 1 min and washed with PBS again. Optical density at 492 nm is determined with a 96-well plate spectrometer reader. For each time point, an aliquot without biocide is a positive control. Percent inhibition of biofilm accumulation can be determined from the formula (A₄₉₂ₚₒₛᵢₜᵢᵥₑ−A_{492biocide})/(A₄₉₂ₚₒₛᵢₜᵢᵥₑ−A₄₉₂)×100%, Such plating methods are well known to one skilled in the art and could be modified as desired for analysis of the biofilm.

For determination of the bacterial resistance of the biofilm formed on the catheter, the method described above was repeated using the same conditions except that the inoculated fluid continuously flowed through the catheter for two hours before fluid flow was discontinued to form a biofilm on the catheter.

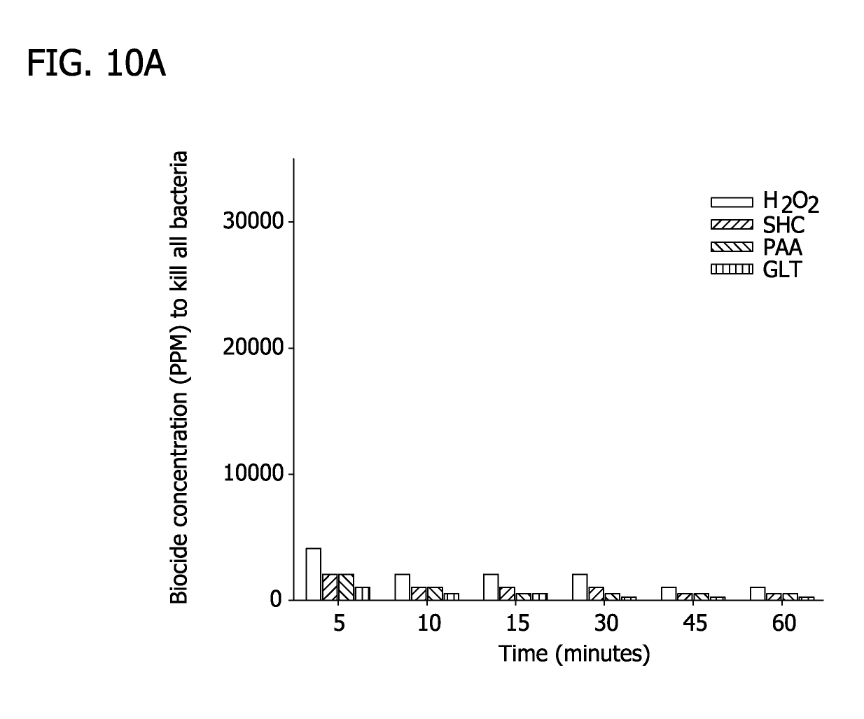

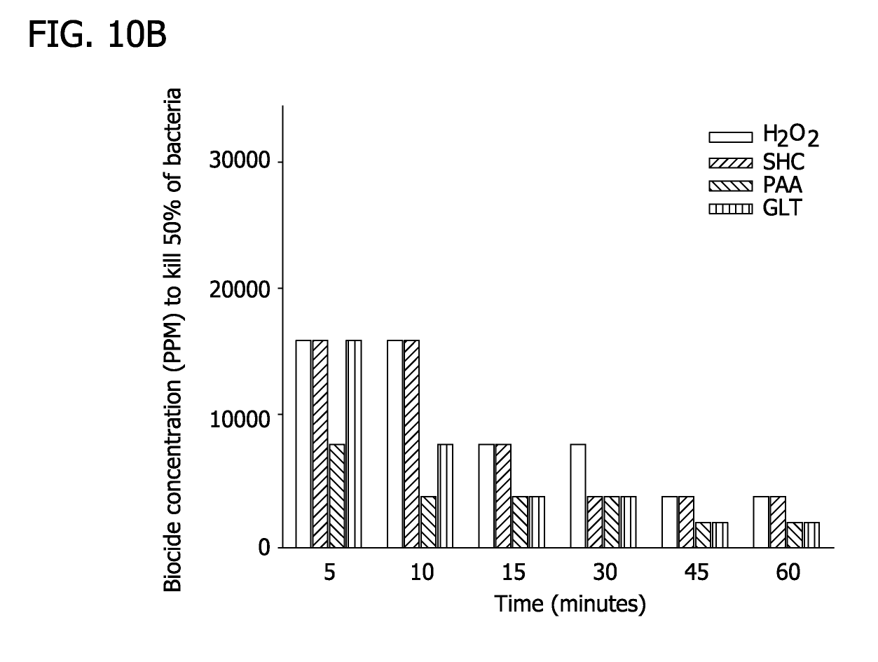

The results of this study are shown in FIGS. 10A-B, which depict the biocide concentration (in ppm) required to kill 100% of the bacteria present as planktonic cells (FIG. 10A) or 50% of bacteria that grew in the biofilm within a period of time ranging from 5 to 60 minutes (FIG. 10B). In all cases, significantly more biocide was required to kill biofilm as compared to planktonic cells. PAA was most effective for biofilm kill, and GLT was most effective in killing planktonic cells.

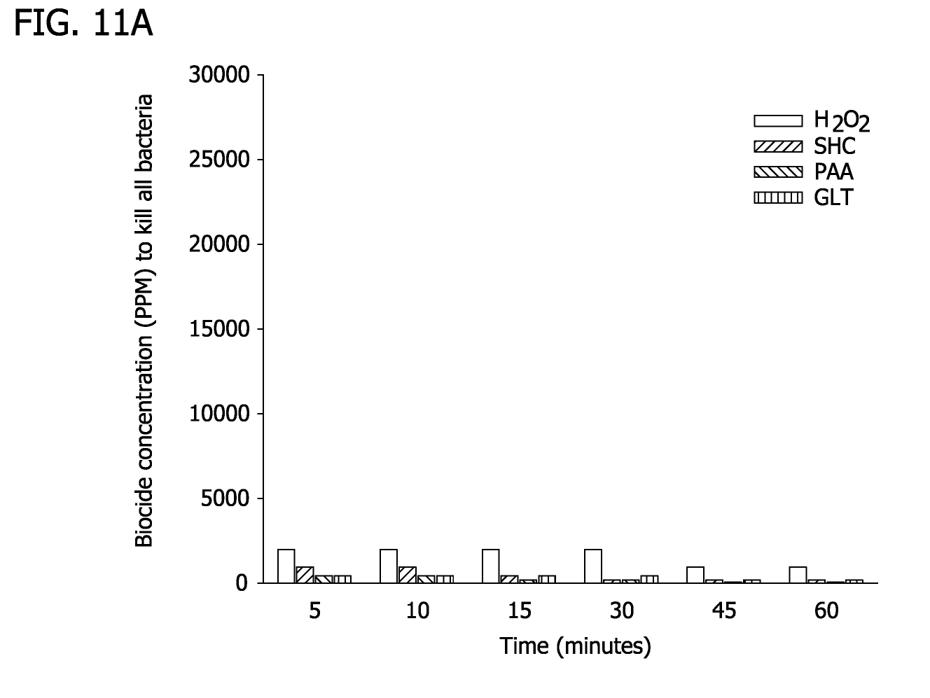

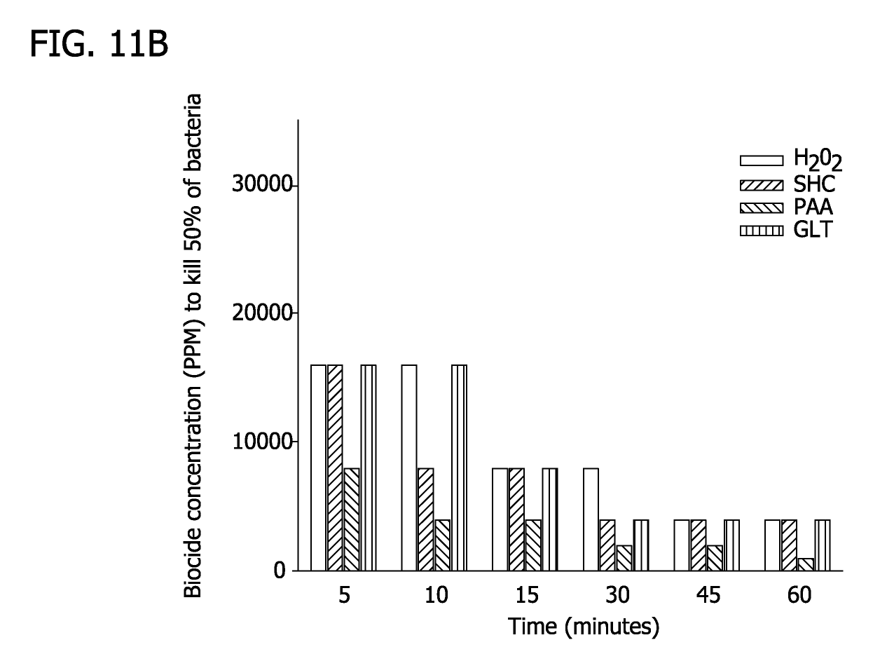

The experiment was repeated as described above, except that Burkholderia cepacia (1×10⁹ CFU/ml) was used to inoculate the serum. FIG. 11B depicts the results of that study, in which hydrogen peroxide, SHC and GLT had nearly identical effect on biofilm kill and PAA was most effective in killing biofilms. For planktonic cell kill, SHC, PAA and GLT performed comparably and were more effective than hydrogen peroxide (FIG. 11A).

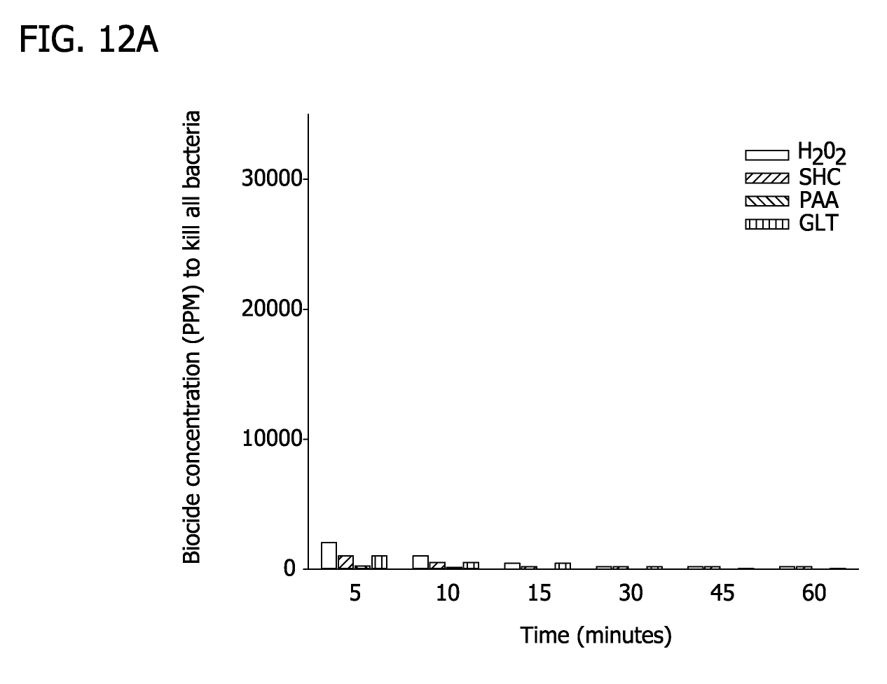

The experiment was repeated as described above, except that Escherichia coli (1×10⁹ CFU/ml) was used to inoculate the serum. FIGS. 12A-B depict the results of that study, in which all biocides were comparable in planktonic cell kill at the concentrations tested, but PAA was most effective in biofilm kill over all time periods. GLT was least effective in E. coli biofilm kill.

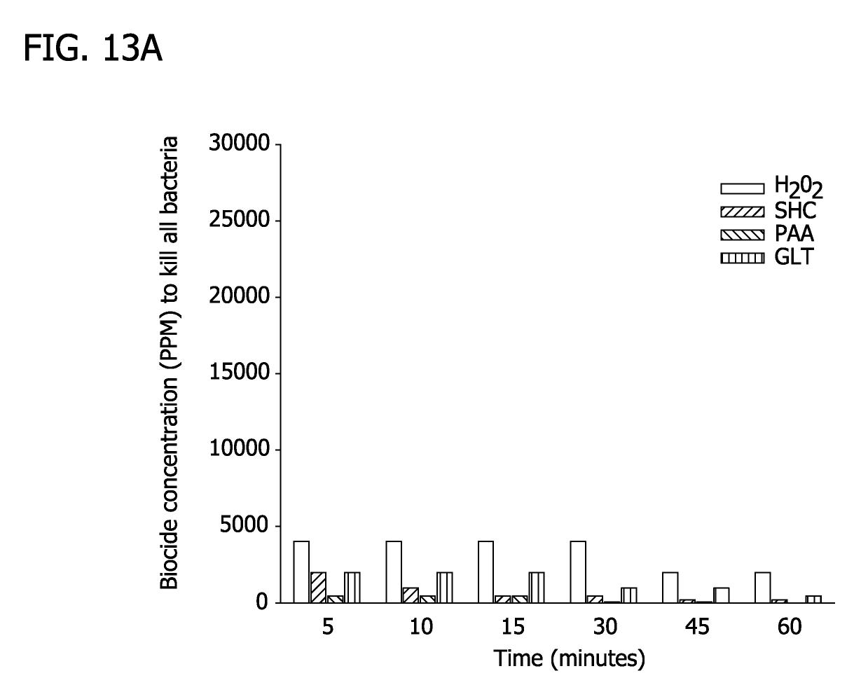

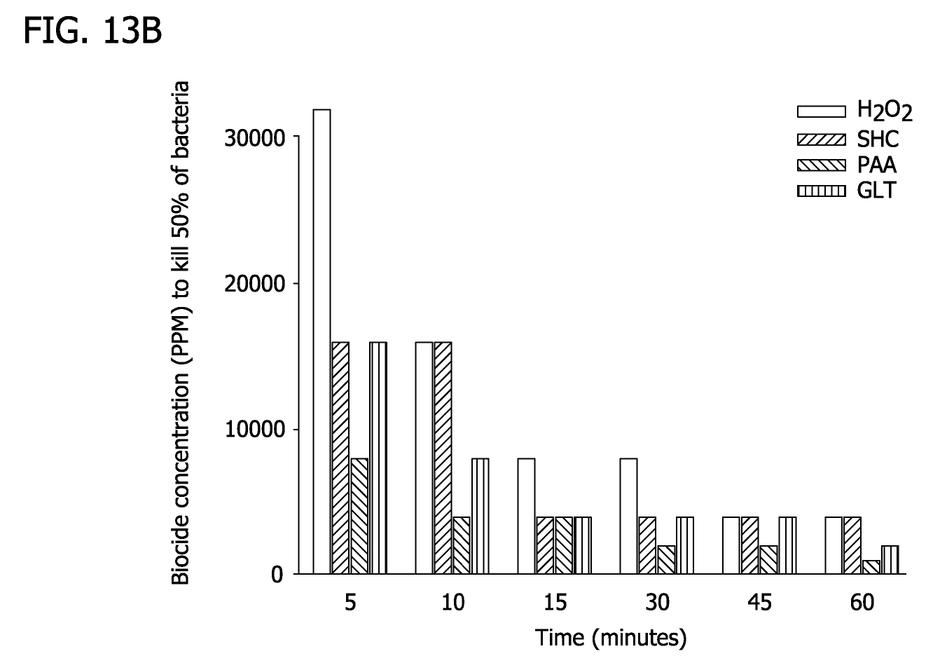

The experiment was repeated as described above, except that Enterococcus faecalis (1×10⁹ CFU/ml) was used to inoculate the serum. FIGS. 13A-B depict the results of that study, in which PAA was most effective in planktonic cell and biofilm kill. Hydrogen peroxide was relatively uneffective at 5 minutes of treatment as compared to the other biocides.

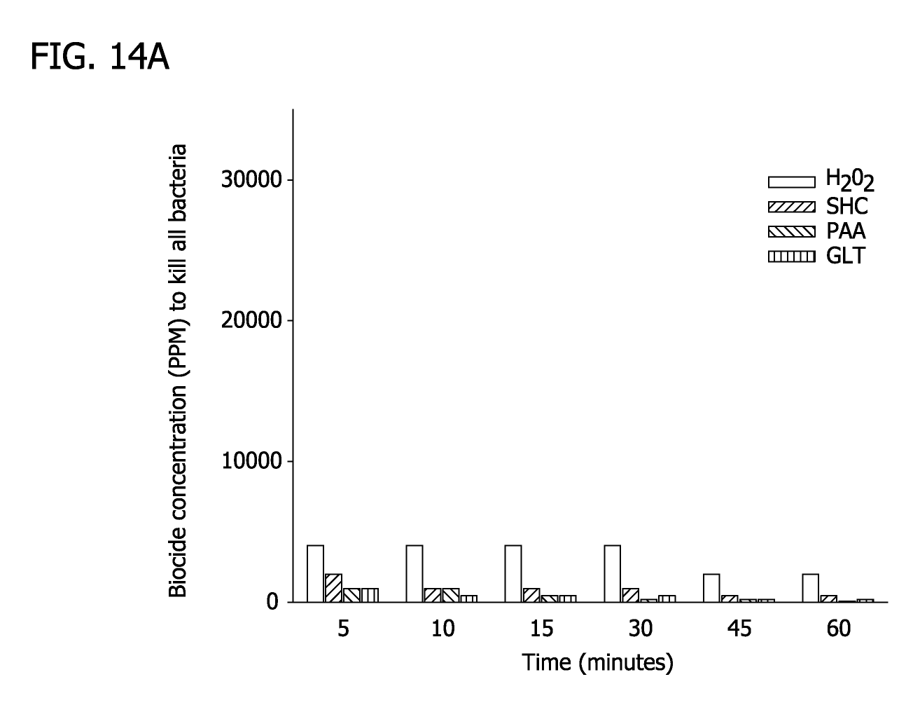

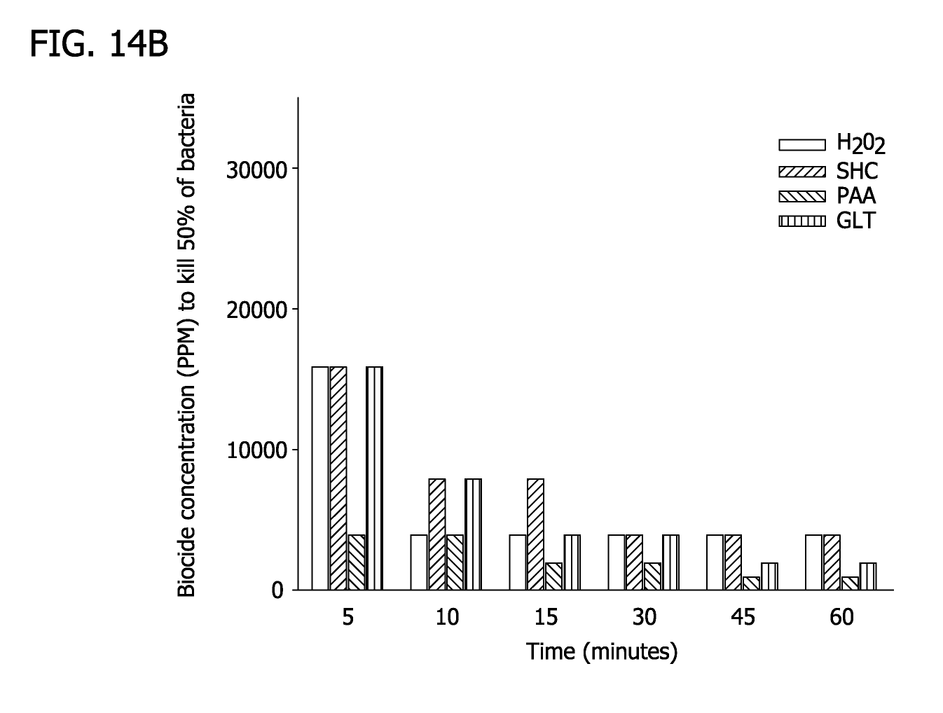

The experiment was repeated as described above, except that methicillin resistant Staphylococcus aureus (MRSA; 1×10⁹ CFU/ml) was used to inoculate the serum. FIGS. 14A-B depict the results of that study, in which PAA, SHC and GLT had similar effect on planktonic cell kill, and PAA was most effective in killing biofilms. For MRSA biofilm kill, PAA>GLT>hydrogen peroxide>SHC.

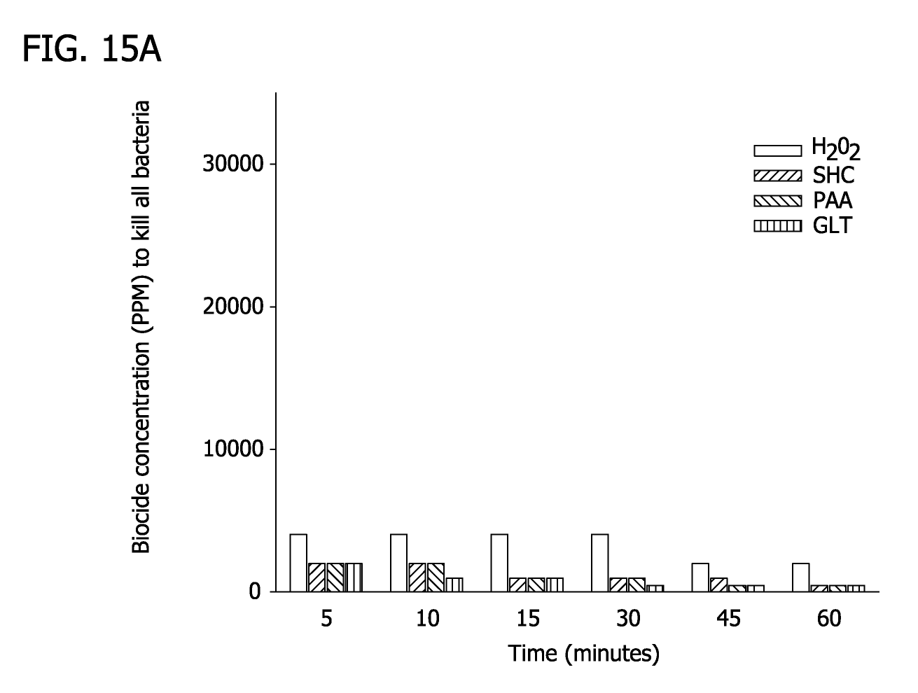

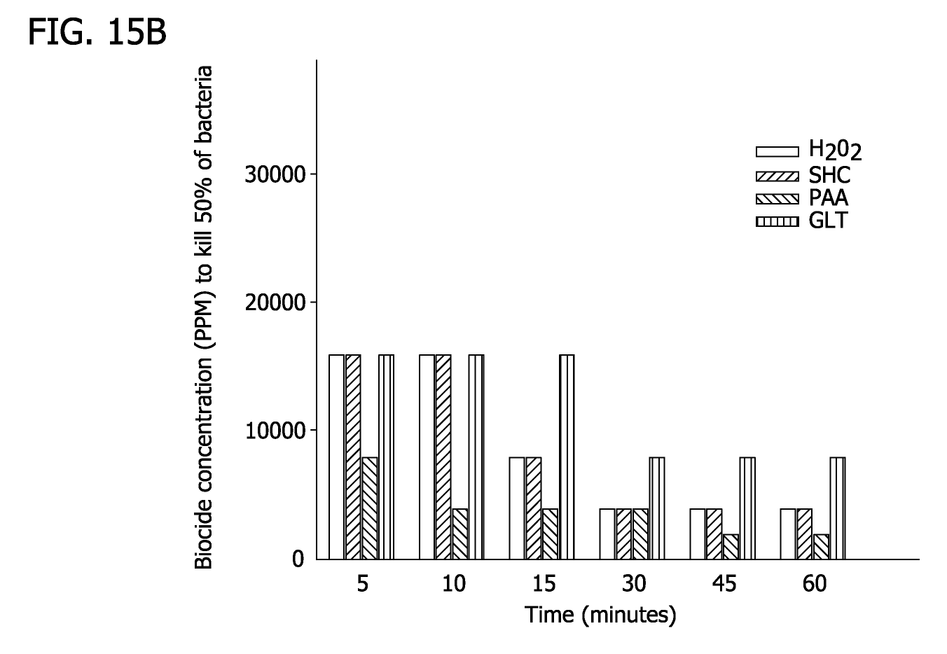

The experiment was repeated as described above, except that Staphylococcus epidermis (1×10⁹ CFU/ml) was used to inoculate the serum. FIGS. 15A-B depict the results of that study, in which SHC, PAA and GLT performed similarly in planktonic cell kill and outperformed hydrogen peroxide. For biofilm kill, PAA was most effective, GLT was least effective, and hydrogen peroxide and SHC performed comparably.

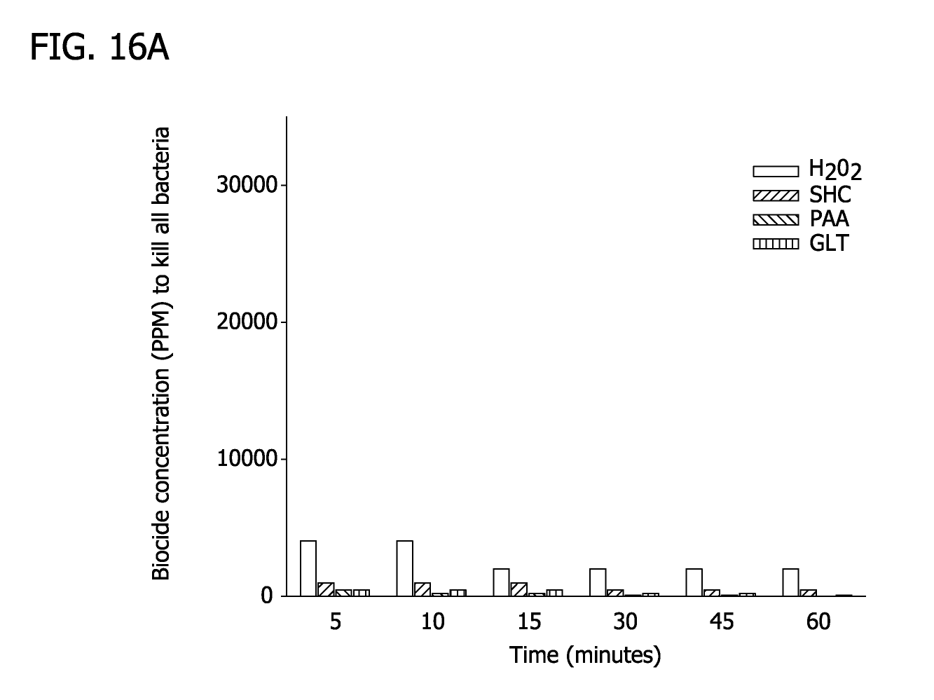

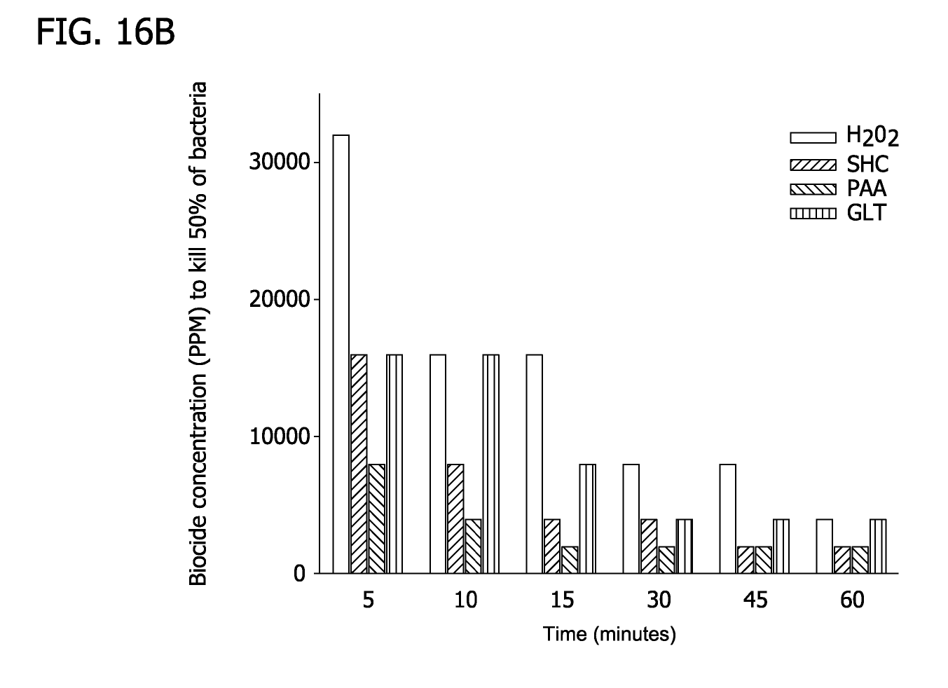

The experiment was repeated as described above, except that Pseudomonas aeruginosa (1×10⁹ CFU/ml) was used to inoculate the serum. FIGS. 16A-B depict the results of that study, in which PAA was most effective in killing biofilms (PAA>SHC>GLT>hydrogen peroxide). For planktonic cell kill, PAA and GLT performed comparably and were more effective than hydrogen peroxide and SHC.

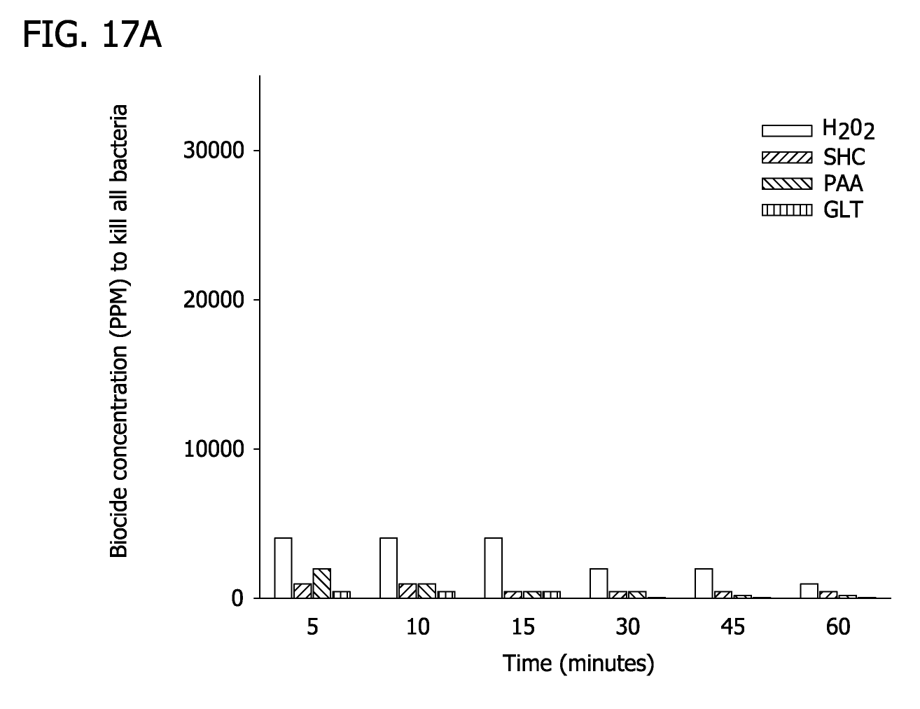

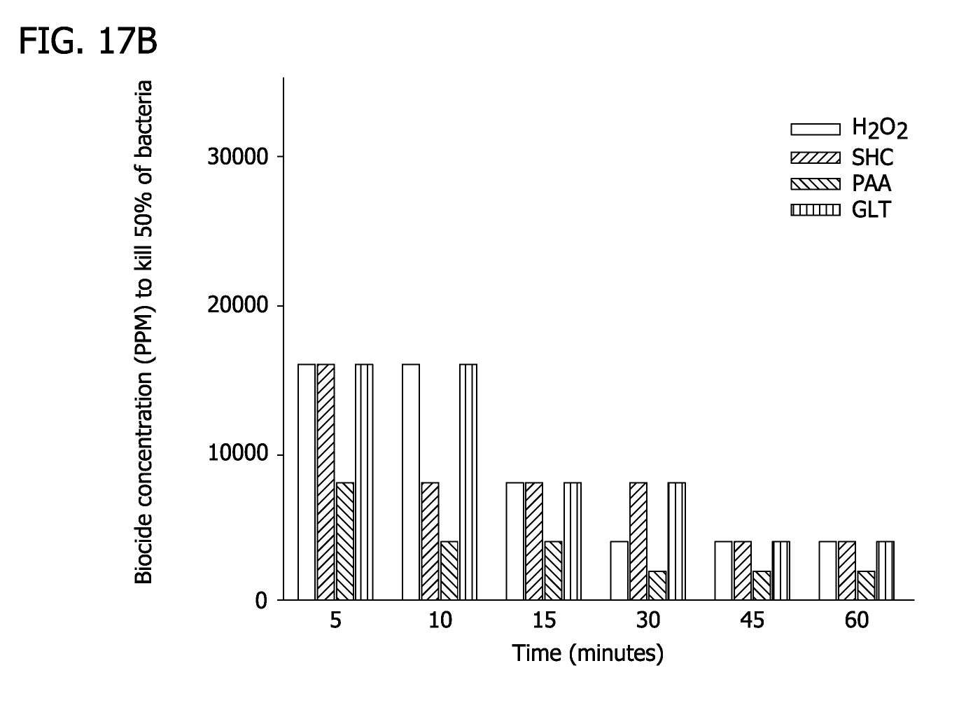

The experiment was repeated as described above, except that Stenotrophomonas maltophilia (1×10⁹ CFU/ml) was used to inoculate the serum. FIGS. 17A-B depict the results of that study, in which GLT was somewhat more effective in killing planktonic cells as compared to SHC or PAA and considerably more effective than hydrogen peroxide. For biofilm kill, hydrogen peroxide, SHC and GLT had nearly identical effect, and PAA was most effective in killing biofilms.

### Example 2

### Evaluation of Mucin in Controlling Colonization of MRSA in Endotracheal Tube Models

A system as shown in FIG. 7 was used in this study. Artificial saliva was placed in a sealed bottle as the second fluid source 609. The first fluid source was provided by an oxygen tank used to saturate the artificial saliva with oxygen. A pump 631 was used to deliver the saliva to an endotracheal tube 3 positioned in the sampling apparatus 1.

Initially, only the second fluid source, saliva inoculated with MRSA (1×10⁹ CFU/ml), was used to contaminate the endotracheal tube. The inoculated fluid continuously flowed through IV tubing directly through the endotracheal tube for 60 minutes to form a biofilm on the tube. Fluid flow was then stopped and the tubing was then reconfigured as shown in FIG. 7 so that oxygen could be introduced. In these test runs, 100 mg/L mucin was added to the saliva. The fluid mixture continuously flowed through the catheter for 48 hours before fluid flow was discontinued to determine the effect of mucin on the bacterial resistance of the biofilm formed on the endotracheal tube. In all, four runs were completed at 0 mg/L mucin (control), 100 mg/L mucin, 500 mg/L mucin and 1000 mg/L mucin. After each run, the endotracheal tube was cut aseptically into one cm segments. The segments were transferred to test tubes containing one ml saline and the adherent cells were dislodges by sonication in an aquasonic device, vortexed for about one minute, and then counted on Tropic soya Agar.

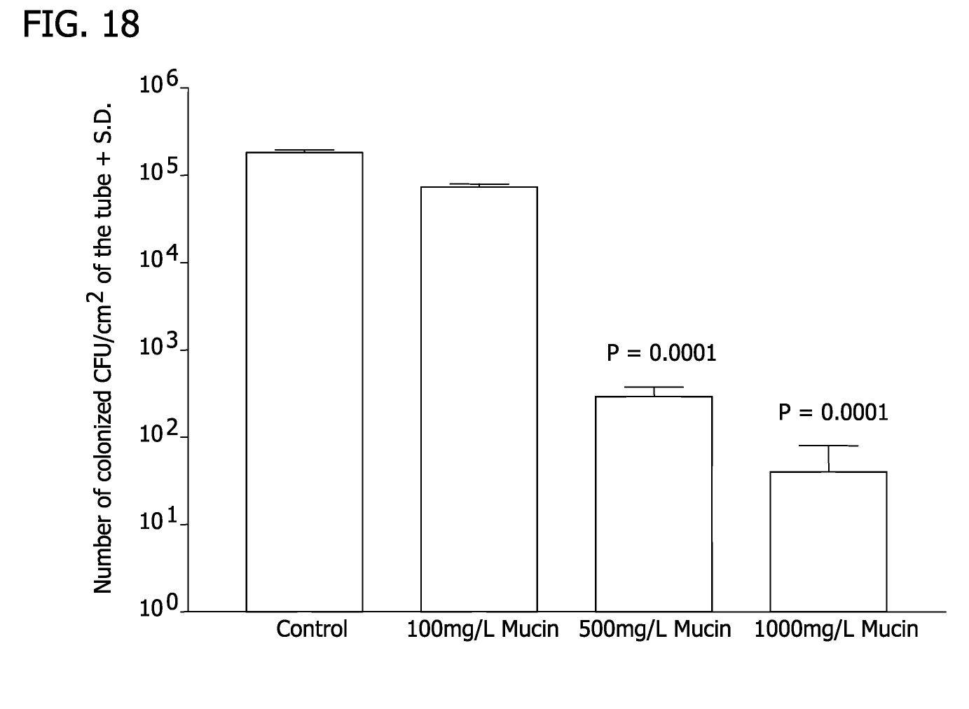

The results of this study are shown in FIG. 18, which depicts the number of MRSA colony forming units per unit of surface area (CFU/cm²) on the tube for each mucin concentration. Mucin was effective in decreasing the colonization of MRSA on endotracheal tubes. The log₁₀ reduction of colonized MRSA was 0.5, 3 (P=0.0001) and 3.5 (P=0.0001) at 100, 500 and 1000 mg/L of mucin, respectively. Mucin significantly decreased colonization of MRSA on endotracheal tubes in vitro. This pathogen plays a significant role in the onset of ventricular associated pneumonia by causing sustained tracheal colonization.

### Example 3

### Effect of Albumin on Colonization of MRSA in Endotracheal Tube Models

A system as shown in FIG. 7 was used in this study. Artificial saliva was placed in a sealed bottle as the second fluid source 609. The first fluid source was provided by an oxygen tank used to saturate the artificial saliva with oxygen. A pump 631 was used to deliver the saliva to an endotracheal tube 3 positioned in the sampling apparatus 1. Each endotracheal tube was pretreated by coating the tube with undiluted bovine serum albumin, except for the untreated tube used as a control.

Initially, only the second fluid source, saliva inoculated with MRSA (1×10⁹ CFU/ml), was used to contaminate the endotracheal tube. The inoculated fluid continuously flowed through IV tubing directly through the endotracheal tube for 60 minutes to form a biofilm on the tube. Fluid flow was then stopped and the tubing was then reconfigured as shown in FIG. 7 so that oxygen could be introduced. In these test runs, 100 mg/L mucin was added to the saliva. The fluid mixture continuously flowed through the catheter for 48 hours before fluid flow was discontinued to determine the effect of albumin on the colonization of MRSA on the endotracheal tube. In all, four runs were completed at 100 mg/L mucin on an uncoated endotracheal tube (control), 0 mg/ml mucin on an endotracheal tube entirely coated with albumin (albumin alone), 100 mg/L mucin on an endotracheal tube with only the inner lumen coated with albumin (inner lumen coat), and 100 mg/L mucin on an endotracheal tube entirely coated with albumin (whole tube coat). After each run, the endotracheal tube was cut aseptically into one cm segments. The segments were transferred to test tubes containing one ml saline and the adherent cells were dislodges by sonication in an aquasonic device, vortexed for about one minute, and then counted on Tropic soya Agar.

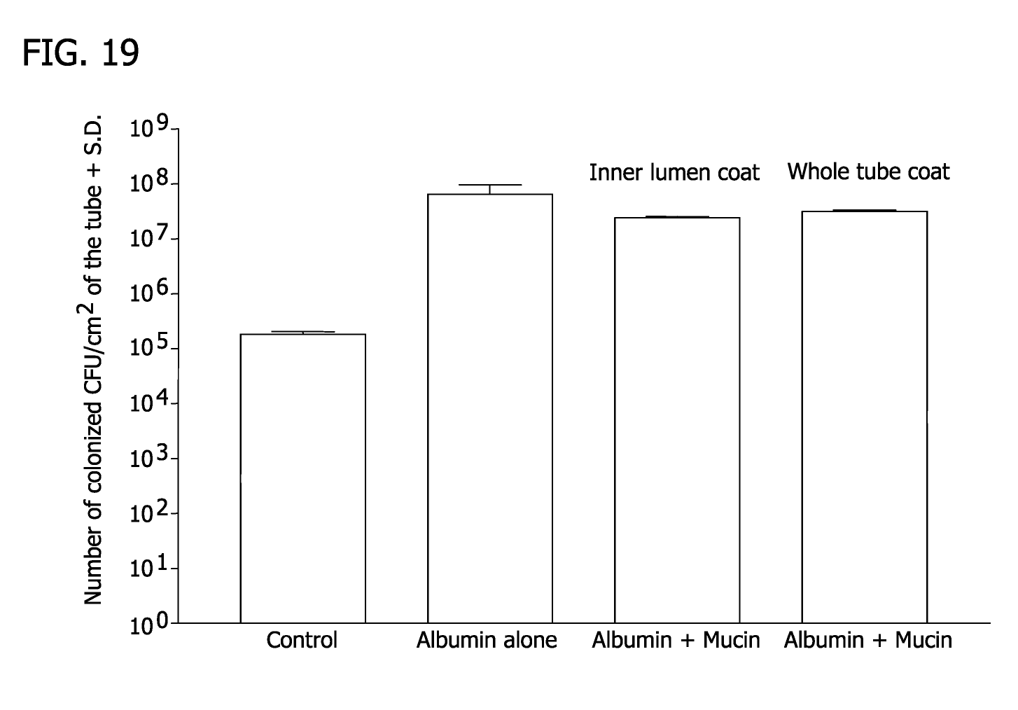

The results of this study are shown in FIG. 19, which depicts the number of MRSA colony forming units per unit of surface area (CFU/cm²) on the tube for each embodiment. MRSA colonization of endotracheal tubes increased significantly as a result of albumin coating. The log₁₀ increase of colonized cells was 2.4 (P<0.0001) with whole tube coating.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions and methods without departing from the scope of the invention, it is intended that all matter contained in the above description and shown in the accompanying drawings shall be interpreted as illustrative and not in a limiting sense.

What is claimed is:

1. An apparatus for forming biofilm on a fluid flow conduit, the apparatus comprising:
a tubular body defining a test chamber having a vertical axis, said body having upper and lower ends;
an upper end closure closing the upper end of the tubular body;
a lower end closure closing the lower end of the tubular body;
an outlet in the tubular body toward the upper end of the body; and
an opening in one of said end closures for receiving a fluid flow conduit such that the conduit extends generally vertically into the test chamber to define an annular space between the conduit and the tubular body whereby fluid passing through said conduit and into the chamber flows into said annular space and exits said outlet.

2. The apparatus as set forth in claim 1 further comprising a pump for pumping fluid from a fluid source to the conduit; an outlet line for delivering fluid from the outlet to a collection vessel; or an opening in the other of said end closures whereby said fluid flow conduit can be selectively placed in either of the two openings, and a removable plug for closing the opening not selected.

3. The apparatus as set forth in claim 2 further comprising a filter in said outlet line for filtering bacteria from the fluid.

4. The apparatus as set forth in claim 1 wherein said opening lies generally on said vertical axis of the chamber.

5. The apparatus as set forth in claim 1 in combination with a fluid flow conduit extending through the upper end closure down into the test chamber or through the lower end closure up into the test chamber.

6. A system comprising the apparatus of claim 1 wherein a fluid flow conduit is in the opening in said one of the end closures and extending into the test chamber, and at least one fluid source for delivery of a fluid to said conduit.

7. A system as set forth in claim 6 wherein at least two fluid sources are connected in series for delivery of a fluid mixture to said conduit.

8. A system as set forth in claim 7 wherein the fluid from at least one of said fluid sources is selected from a group consisting of a gas, an antimicrobial agent and a biofilm formation inhibitor.

9. A system as set forth in claim 6 wherein said opening is a first opening in the upper end closure, the fluid flow conduit in the first opening, and a second opening in the lower end closure through which fluid flowing continuously into the test chamber exits the test chamber.

10. A system as set forth in claim 6 wherein the fluid flow conduit is in the first opening, and wherein said outlet is closed thereby to allow static testing of fluid in the chamber.

11. A process for growing and assaying biofilms on a test device in vitro, the process comprising:
providing a system comprising an apparatus as set forth in claim 1, a fluid flow conduit in the opening in said one of the end closures of said apparatus and extending into the test chamber, and at least one fluid;
passing the fluid through said conduit and into the chamber such that it flows into said annular space, so as to form a biofilm on at least one surface of said conduit;
removing said conduit from said apparatus; and
analyzing the biofilm.

12. The process as set forth in claim 11 wherein said conduit comprises a medical device.

13. The process as set forth in claim 12 wherein the medical device comprises a catheter, a cannula, a vascular graft, a vascular catheter port, a vascular access device, a shunt, a heart valve, an incontinence device, a penile implant, or a tube.

14. The process as set forth in claim 13 wherein the medical device comprises a vascular catheter, a urinary catheter, or an endotracheal tube.

15. The process as set forth in claim 11 wherein the fluid comprises saliva, serum, blood, urine or other human body fluid, or an artificial body fluid which is physiologically acceptable when administered to a human.

16. The process as set forth in claim 11 wherein the fluid comprises saliva and oxygen, and said conduit comprises an endotracheal tube; the fluid comprises serum or blood and said conduit comprises a vascular catheter; or the fluid comprises urine and said conduit comprises a urinary catheter.

17. The process as set forth in claim 11 wherein said conduit is in the first opening, and wherein said outlet is closed thereby to allow static testing of fluid in the chamber.

18. The process as set forth in claim 11 wherein said opening is a first opening in the upper end closure, said conduit is in the first opening, and the fluid flows into the test chamber and exits the test chamber through a second opening in the lower end closure.

19. The process as set forth in claim 11 further comprising physically removing the biofilm from said conduit or at least one portion of said conduit and assaying the removed biofilm.

20. The process as set forth in claim 19 further comprising cutting said conduit into segments after said conduit is removed from said apparatus; placing the segments into a container; adding a buffer solution to the container; physically removing the biofilm via sonication and vortexing, and assaying the removed biofilm for the number of organisms in the biofilm.

21. The process as set forth in claim 11 wherein said conduit is treated with an antibiotic or a biofilm formation inhibitor before passing the fluid through said conduit to expose said conduit to biofilm forming organisms; and the biofilm is analyzed by assaying the number of organisms in the biofilm.

22. The process as set forth in claim 21 wherein said conduit is treated by coating said conduit with a solution of the antimicrobial agent or biofilm formation inhibitor.

23. The process as set forth in claim 21 wherein the antimicrobial agent or biofilm formation inhibitor comprises a glycoprotein, a polysaccharide, a non-steroidal anti-inflammatory drug (NSAID), tetracycline, rifamycin, a macrolide, penicillin, cephalosporin, a beta-lactam antibiotic, an aminoglycoside, chloramphenicol, a sulfonamide, a glycopeptide, a quinolone, fusidic acid, trimethoprim, metronidazole, clindamycin, mupirocin, a polyene, an azole, a benzalkonium halide, a silver salt, a beta-lactam inhibitor, triclosan, chlorhexidine, nitrofurazone, rifampin, gentamycin, minocyclin, imipenem, aztreonam, sulbactam, or a chelating agent.

24. The process as set forth in claim 21 wherein the biofilm formation inhibitor comprises EDTA (ethylenediaminetetraacetic acid), EGTA (O,O′-bis(2-aminoethyl)ethyleneglycol-N,N,N′,N′-tetraacetic acid), salicylic acid or a salt thereof, mucin, or chitosan.

25. The process as set forth in claim 24 wherein the biofilm formation inhibitor comprises mucin.
